(19) United States
(12) Patent Application Publication
(10) Pub. No.: US 2026/0364161 A1
(43) Pub. Date: Nov. 19, 2026

(54) VACCINE

(71) Applicant: GLAXOSMITHKLINE BIOLOGICALS SA, Rixensart (BE)

(72) Inventors: Danilo GOMES MORIEL, Siena (IT); Margherita COCCIA, Rixensart (BE); Emilia CAPPELLETTI, Siena (IT); Anna Maria Rosaria COLUCCI, Siena (IT)

(73) Assignee: GLAXOSMITHKLINE BIOLOGICALS SA, Rixensart (BE)

(21) Appl. No.: 19/121,510

(22) PCT Filed: Oct. 17, 2023
(86) PCT No.: PCT/EP2023/078896

§ 371 Date: Apr. 16, 2025

(30) Foreign Application Priority Data
Oct. 18, 2022 (GB) 2215414.0

(51) Int. Cl. A61K 39/09; A61K 39/00; A61K 39/39; A61P 31/04

(52) U.S. Cl. CPC A61K 39/092; A61K 39/39; A61P 31/04; A61K 2039/545; A61K 2039/55505; A61K 2039/55511; A61K 2039/575; A61K 2039/6037

(57) ABSTRACT

The present invention relates to compositions comprising at least one Group A Streptococcus antigen, an aluminium salt and (a) a TLR7 agonist, or (b) a benzonapthyridine compound. The present invention further relates to methods of making an immunogenic composition comprising at least one antigen, an aluminium salt and (a) a TLR7 agonist, and/or (b) a benzonapthyridine compound. The present invention also relates to vaccines comprising the compositions, and methods of using and uses of the compositions.

### FIELD OF THE INVENTION

The present invention relates to compositions comprising antigens, aluminium salts and TLR7 agonists and/or benzonapthyridine compounds. The present invention also relates to methods of making such compositions, vaccines comprising the compositions, and methods of using or uses of the compositions.

### BACKGROUND TO THE INVENTION

Group A Streptococcus (GAS) causes a diverse spectrum of diseases, from superficial infections (pharyngitis, skin infections) to severe invasive diseases (cellulitis, puerperal sepsis, necrotizing fasciitis, streptococcal toxic shock syndrome), with a high frequency of serious autoimmune sequelae in low- and middle-income countries (LMICs), such as acute rheumatic fever, (ARF); rheumatic heart disease, (RHD), and glomerulonephritis (Ralph et al.; Curr. Top. Microbiol. Immunol. 2013; 368:1-27).

Pharyngitis is the most frequent symptomatic GAS infection in children across the world, with more than 600 million cases estimated annually (Carapetis et al.; The Lancet 2005; 5:685-94) and is an important driver of antibiotic use (Dooling et al.; Jama Pediatr. 2014; 168:1073-4) that can ultimately result in increased antimicrobial resistance, a growing public health crisis (Jansen et al.; Nature Medicine 2018; 24:10-19). Pharyngitis could lead to RHD, which is a chronic inflammatory heart valve condition representing the main global burden of GAS. In 2015, 319 thousand deaths due to RHD were estimated, with >33 million RHD cases and 10 million disability-adjusted life-years (DALYs) lost (Watkins et al.; N. Engl. J. Med. 2017; 377:713-722). Vaccination is the most practical strategy to reduce global GAS associated disease burden in the long term. However, no commercial vaccine is still available against this pathogen (Vekemans et al.; Clin. Infect. Dis. 2019; 69: 877-883).

Group A Carbohydrate (GAC) is a surface polysaccharide comprising of a polyrhamnose backbone with alternating N-acetylglucosamine (GlcNAc) at the side chain. It represents an attractive vaccine candidate as it is highly conserved and expressed across GAS strains. Indeed, one of the major obstacles for vaccine strategy development is represented by GAS serotype diversity related to other non-carbohydrate antigens (Walker et al.; Clin. Microbiol. Rev. 2014; 27:264-301).

It has been reported that human anti-GAC sera successfully promoted phagocytosis of several GAS strains (Salvadori et al.; J. Infect. Dis. 1995; 171:593-600), while mice immunized with GAC conjugated to tetanus toxoid (TT) or CRM197 carrier proteins were protected against GAS challenges (Kabanova et al.; Vaccine 2010; 29:104-114 and Sabharwal et al.; J Infect. Dis. 2006; 193:129-135). Moreover, an inverse relationship between high anti-GAC antibody titers and the presence of GAS in the throat of Mexican children was evidenced (Sabhawral et al. as referenced above).

Conserved protein antigens are also in vaccine development against GAS. In particular, Streptolysin O (SLO), SpyAD and SpyCEP were identified as promising vaccine candidates through a reverse vaccinology approach (Bensi et al.; Mol. Cell Proteomics 2012; 11, M111 015693). SLO has been shown to be a key virulence factor of GAS by preventing internalization of the bacteria into lysosomes where they can be destroyed (Cunningham; Clin. Microbiol. Rev. 2000; 13:470-511). Moreover, SLO promotes GAS resistance to phagocytic clearance by neutrophils, facilitating GAS escape from innate immune killing, and an inactivated SLO was demonstrated to be protective in a murine model against GAS challenge (Vchiyama et al.; Front Immunol. 2015; 6:581). SpyAD is a surface-exposed adhesin that mediates GAS interaction with host cells. Moreover, deletion of the SpyAD gene in a GAS strain led to an impaired capacity of the knockout mutant to properly divide, suggesting also an important role in bacterial division (Gallotta et al.; Infect. Immun. 2014; 82:2890-2901). Finally, SpyCEP is a multi-domain proteinase, with a catalytic domain responsible for the interleukin (IL)-8 and other chemokines cleavage. Cleavage of IL-8 represents a mechanism of immune evasion, preventing IL-8 C-terminus-mediated endothelial translocation and subsequent recruitment of neutrophils (Edwards et al.; J. Infect. Dis. 2005; 192:783-790 and Jobichen et al.; J. Infect. Dis. 2005; 192:783-790).

These three protein antigens are highly conserved and prevalent in clinical collections and, together with GAC, could virtually cover all GAS clinical isolates (Davies et al.; Nat. Genet. 2019; 51:1035-1043). Thus, the formulation of a multicomponent vaccine composed of recombinant SLO, SpyAD and SpyCEP with GAC-CRM₁₉₇ conjugate has been proposed (Vekemans et al. as referenced above).

Multivalent vaccines comprising recombinant SLO, SpyAD, SpyCEP and GAC-CRM₁₉₇ have the potential to be excellent vaccines. However, it is an aim of the present invention to yet further improve the immune responses generated to vaccines comprising recombinant SLO, SpyAD, SpyCEP and GAC-CRM₁₉₇ antigens.

### SUMMARY OF THE INVENTION

The present inventors have surprisingly found that a composition comprising at least one Group A Streptococcus antigen, an aluminium salt (such as aluminium hydroxide) and a TLR7 agonist and/or benzonapthyridine compound (such as LHD153R) is not only significantly more immunogenic than a corresponding composition lacking the TLR7 agonist and/or benzonapthyridine compound but when administered to mice elicits significantly more functional antibodies against the at least one Group A Streptococcus antigen compared to a corresponding composition lacking the TLR7 agonist and/or benzonapthyridine compound.

Furthermore, the present inventors have surprisingly found that such a composition may be advantageously prepared using a method comprising preparing a mixture of:
(i) the at least one antigen pre-absorbed on the aluminium salt and the TLR7 agonist and/or benzonapthyridine compound;
(ii) the at least one antigen and the TLR7 agonist and/or benzonapthyridine compound pre-absorbed on the aluminium salt; or
(iii) the at least one antigen pre-absorbed on the aluminium salt and the TLR7 agonist and/or benzonapthyridine compound pre-absorbed on the aluminium salt.

Accordingly, in a first aspect of the invention, there is provided an immunogenic composition comprising at least one Group A Streptococcus antigen, an aluminium salt and a TLR7 agonist.

In a second aspect of the invention, there is provided an immunogenic composition comprising at least one Group A Streptococcus antigen, an aluminium salt and a benzonapthyridine compound.

In a third aspect of the invention, there is provided a method of making an immunogenic composition comprising at least one antigen, an aluminium salt and (a) a TLR7 agonist, and/or (b) a benzonapthyridine compound comprising preparing a mixture of:
(i) the at least one antigen pre-absorbed on the aluminium salt and the TLR7 agonist and/or benzonapthyridine compound;
(ii) the at least one antigen and the TLR7 agonist and/or benzonapthyridine compound pre-absorbed on the aluminium salt; or
(iii) the at least one antigen pre-absorbed on the aluminium salt and the TLR7 agonist and/or benzonapthyridine compound pre-absorbed on the aluminium salt.

In a fourth aspect, there is provided an immunogenic composition that is obtained or obtainable from the methods of the invention.

In a fifth aspect of the invention, there is provided a vaccine comprising the immunogenic composition of the invention.

In a sixth aspect of the invention, there is provided the immunogenic composition or vaccine or the invention for use in a method of preventing infection by and/or preventing autoimmune disease following infection by Group A Streptococcus.

In a seventh aspect of the invention, there is provided a method of preventing infection by and/or preventing autoimmune disease following infection by Group A Streptococcus comprising administering an effective amount of the immunogenic composition or vaccine of the invention.

In an eighth aspect of the invention, there is provided a use of the immunogenic composition or vaccine of the invention for preparing a medicament for use in a method of preventing infection by and/or preventing autoimmune disease following infection by Group A Streptococcus.

### BRIEF DESCRIPTION OF THE FIGURES

FIG. 1 provides graphs presenting data on the IgG response seen in mice after vaccination with vaccines comprising Streptococcus A antigens, aluminium hydroxide and LHD153R or vaccines comprising Streptococcus A antigens and aluminium hydroxide without LHD153R. The Streptococcus A antigens were administered at three different doses (low, medium and high). The low dose corresponds to 78 ng of SpyAD polypeptide, 78 ng of SLO polypeptide, 78 ng of SpyCEP polypeptide and 39 ng of GAC-CRM197. The medium dose corresponds to 625 ng of SpyAD polypeptide, 625 ng of SLO polypeptide, 625 ng of SpyCEP polypeptide and 313 ng of GAC-CRM197. The high dose corresponds to 5 000 ng of SpyAD polypeptide, 5 000 ng of SLO polypeptide, 5 000 ng of SpyCEP polypeptide and 2 500 ng of GAC-CRM197. Panel A depicts the antibody response (measured in RLU/ml) to SpyCEP at 27 days post-vaccination, Panel B depicts the antibody response (measured in RLU/ml) to SpyCEP at 42 days post-vaccination, Panel C depicts the antibody response (measured in RLU/ml) to SpyAD at 27 days post-vaccination, Panel D depicts the antibody response (measured in RLU/ml) to SpyAD at 42 days post-vaccination, Panel E depicts the antibody response (measured in RLU/ml) to SLO at 27 days post-vaccination, Panel F depicts the antibody response (measured in RLU/ml) to SLO at 42 days post-vaccination, Panel G depicts the antibody response (measured in RLU/ml) to GAC at 27 days post-vaccination and Panel H depicts the antibody response (measured in RLU/ml) to GAC at 42 days post-vaccination.

FIG. 2 provides graphs presenting data on the level of functional antibodies seen in mice seen in mice after vaccination with vaccines comprising Streptococcus A antigens, aluminium hydroxide and LHD153R or vaccines comprising Streptococcus A antigens and aluminium hydroxide without LHD153R. The Streptococcus A antigens were administered at two different doses (medium and high). The medium dose corresponds to 625 ng of SpyAD polypeptide, 625 ng of SLO polypeptide, 625 ng of SpyCEP polypeptide and 313 ng of GAC-CRM197. The high dose corresponds to 5 000 ng of SpyAD polypeptide, 5 000 ng of SLO polypeptide, 5 000 ng of SpyCEP polypeptide and 2 500 ng of GAC-CRM197. Panel A depicts the level of functional antibodies raised against SpyCEP as determined using an IL-8 cleavage assay. Panel B depicts the level of functional antibodies raised against SLO as determined using a hemolysis assay.

FIG. 3 provides graphs presenting data on the ratio of IgG2/IgG1 and IgG3/IgG1 seen in mice after vaccination with vaccines comprising Streptococcus A antigens, aluminium hydroxide and LHD153R or vaccines comprising Streptococcus A antigens and aluminium hydroxide without LHD153R. The dose of Streptococcus A antigens used was 5 000 ng of SpyAD polypeptide, 5 000 ng of SLO polypeptide, 5 000 ng of SpyCEP polypeptide and 2500 ng of GAC-CRM197. For each panel, the left hand column corresponds to a vaccine comprising Streptococcus A antigens and aluminium hydroxide only, the central column corresponds to a vaccine comprising Streptococcus A antigens, aluminium hydroxide and LHD153R, and the right hand column corresponds to a vaccine comprising Streptococcus A antigens and no adjuvant. Panel A describes the ratio of IgG2/IgG1 ratio of antibodies raised against GAC. Panel B describes the ratio of IgG2/IgG1 ratio of antibodies raised against SLO. Panel C describes the ratio of IgG2/IgG1 ratio of antibodies raised against SpyAD. Panel D describes the ratio of IgG2/IgG1 ratio of antibodies raised against SpyCEP. Panel E describes the ratio of IgG3/IgG1 ratio of antibodies raised against GAC. Panel F describes the ratio of IgG3/IgG1 ratio of antibodies raised against SLO. Panel G describes the ratio of IgG3/IgG1 ratio of antibodies raised against SpyAD. Panel H describes the ratio of IgG3/IgG1 ratio of antibodies raised against SpyCEP.

FIG. 4 provides graphs presenting data on the IgG response seen in mice after vaccination with vaccines comprising Streptococcus A antigens, aluminium hydroxide and LHD153R or Streptococcus A antigens with no adjuvant. The Streptococcus A antigens were administered at three different doses (low, medium and high). The low dose corresponds to 78 ng of SpyAD polypeptide, 78 ng of SLO polypeptide, 78 ng of SpyCEP polypeptide and 39 ng of GAC-CRM197. The medium dose corresponds to 625 ng of SpyAD polypeptide, 625 ng of SLO polypeptide, 625 ng of SpyCEP polypeptide and 313 ng of GAC-CRM197. The high dose corresponds to 5 000 ng of SpyAD polypeptide, 5 000 ng of SLO polypeptide, 5 000 ng of SpyCEP polypeptide and 2 500 ng of GAC-CRM197. Panel A depicts the antibody response (measured in RLU/ml) to SpyCEP at 27 days post-vaccination, Panel B depicts the antibody response (measured in RLU/ml) to SpyCEP at 42 days post-vaccination, Panel C depicts the antibody response (measured in RLU/ml) to SpyAD at 27 days post-vaccination, Panel D depicts the antibody response (measured in RLU/ml) to SpyAD at 42 days post-vaccination, Panel E depicts the antibody response (measured in RLU/ml) to SLO at 27 days post-vaccination, Panel F depicts the antibody response (measured in RLU/ml) to SLO at 42 days post-vaccination, Panel G depicts the antibody response (measured in RLU/ml) to GAC at 27 days post-vaccination and Panel H depicts the antibody response (measured in RLU/ml) to GAC at 42 days post-vaccination.

FIG. 5 provides graphs presenting data on the level of functional antibodies seen in mice seen in mice after vaccination with vaccines comprising Streptococcus A antigens, aluminium hydroxide, and LHD153R or Streptococcus A antigens with no adjuvant. The Streptococcus A antigens were administered at two different doses (medium and high). The medium ng dose corresponds to 625 ng of SpyAD polypeptide, 625 ng of SLO polypeptide, 625 ng of SpyCEP polypeptide and 313 ng of GAC-CRM197. The high dose corresponds to 5 000 ng of SpyAD polypeptide, 5 000 ng of SLO polypeptide, 5 000 ng of SpyCEP polypeptide and 2 500 ng of GAC-CRM197. Panel A depicts the level of functional antibodies raised against SpyCEP as determined using an IL-8 cleavage assay. Panel B depicts the level of functional antibodies raised against SLO as determined using a hemolysis assay.

FIG. 6 is a sequence listing.

### GENERAL DEFINITIONS

Unless defined otherwise, technical and scientific terms used herein have the same meaning as commonly understood by a person skilled in the art to which this invention belongs.

In general, the term “comprising” is intended to mean including but not limited to. For example, the phrase “An immunogenic composition comprising at least one Group A Streptococcus antigen, an aluminium salt and a TLR7 agonist” should be interpreted to mean that the composition comprises at least one Group A Streptococcus antigen an aluminium salt and a TLR7 agonist, but the composition may comprise further components.

In some embodiments of the invention, the word “comprising” is replaced with the phrase “consisting of”. The term “consisting of” is intended to be limiting. For example, the phrase “An immunogenic composition consisting of at least one Group A Streptococcus antigen, an aluminium salt and a TLR7 agonist” should be understood to mean that the composition contains at least one Group A Streptococcus antigen, an aluminium salt and a TLR7 agonist and no further components.

In some embodiments of the invention, the word “comprising” is replaced with the phrase “consisting essentially of”. The term “consisting essentially of” means that specific further components can be present, namely those not materially affecting the essential characteristics of the subject matter. For example, the phrase “An immunogenic composition consisting essentially of at least one Group A Streptococcus antigen, an aluminium salt and a TLR7 agonist” may comprise components like buffers or other pharmaceutically acceptable excipients.

The term “about” or “around” when referring to a value refers to that value but within a reasonable degree of scientific error. Optionally, a value is “about x” or “around x” if it is within 10%, within 5%, or within 1% of x.

The singular forms “a”, “an”, and “the” include plural referents unless the content clearly dictates otherwise.

Amino acids “corresponding to” specified positions of a specified SEQ ID NO may be amino acids at the specified positions of the particular SEQ ID NO recited. For example, the amino acid “corresponding to position 272 of SEQ ID NO: 1” may be the amino acid at position 272 of SEQ ID NO: 1. Alternatively, amino acids “corresponding to” positions of a specified SEQ ID NO may be amino acids from an alternative amino acid sequence which correspond to the specified positions of the specified SEQ ID NO. For example, the amino acid “corresponding to position 272 of SEQ ID NO: 1” may be the amino acid from an alternative amino acid sequence which corresponds to position 272 of SEQ ID NO: 1. It is within the capabilities of the person skilled in the art to determine which amino acids in an alternative amino acid sequence “correspond to” the specified positions in the specified SEQ ID NO. For example, the person skilled in the art merely needs to perform a sequence alignment of the alternative amino acid sequence with the specified SEQ ID NO using a suitable alignment algorithm such as that of Needleman and Wunsch described herein, and determine which region of the alternative amino acid sequence aligns to the specified positions in the specified SEQ ID NO. For example, the skilled person is able to align the alternative amino acid sequence with SEQ ID NO: 1 and determine which amino acid aligns, and therefore corresponds to, e.g. position 272 of SEQ ID NO. 1.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotides or amino acid residues at each position are then compared. When a position in the first sequence is occupied by the same nucleotide or amino acid at the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 1, SEQ ID NO: 1 would be the reference sequence. To assess whether a sequence is at least 95% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 95% of the positions are identical, the test sequence is at least 95% identical to SEQ ID NO: 1. If the test sequence is shorter than SEQ ID NO: 1, the gaps or missing positions should be considered to be non-identical positions.

The skilled person is aware of different computer programs that are available to align two sequences. For instance, an alignment between two sequences can be accomplished using a mathematical algorithm. In an embodiment, the two amino acid or nucleic acid sequences are aligned using the Needleman and Wunsch (1970) algorithm which has been incorporated into the GAP program in the Accelrys GCG software package using either a Blosum 62 matrix or a PAM250 matrix, and a gap weight of 16, 14, 12, 10, 8, 6, or 4 and a length weight of 1, 2, 3, 4, 5, or 6.

All publications, patents and patent applications cited herein, whether supra or infra, are hereby incorporated by reference in their entirety.

### At Least One Antigen

The present invention provides an immunogenic composition comprising at least one Group A Streptococcus antigen. Similarly, the present invention provides methods of making an immunogenic composition comprising at least one antigen.

The at least one antigen may comprise an antigen from a bacterium selected from the group consisting of Actinomyces (e.g., A. israelii), Bacillus (e.g., B. anthracis or B. cereus), Bartonella (e.g., B. henselae, or B. quintana), Bordetella (e.g., B. pertussis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. Typhi, S. Enteritidis, S. Paratyphi, S. Typhimurium, or S. Choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).

The at least one antigen may comprise at least one Group A Streptococcus antigen. Herein, the term “Group A Streptococcus” is intended to refer to and is synonymous with the term “Streptococcus pyogenes”. Thus, any instance of the term “Group A Streptococcus” may be replaced with the term “Streptococcus pyogenes”. Streptococcus pyogenes is a species of pathogenic Gram-positive bacteria in the genus Streptococcus. Methods of determining whether a bacterium is Streptococcus pyogenes are well known to the skilled person. Specifically, all Streptococcus pyogenes strains express GAC, and the skilled person is aware of methods for detecting GAC.

Antigens from Streptococcus pyogenes include the antigens disclosed in WO2005032582, WO2006042027, WO2009034473, WO2009081274, and WO2010076618. Suitable antigens include a polysaccharide such as GAC, optionally as part of a polysaccharide-protein conjugate, a SpyCEP polypeptide, a SpyAD polypeptide and/or a SLO polypeptide.

Optionally, the at least one Group A Streptococcus antigen comprises at least one recombinant polypeptide. Optionally, the SpyCEP polypeptide, the SpyAD polypeptide and/or a SLO polypeptide is/are a recombinant polypeptide(s).

Optionally, the at least one Group A Streptococcus antigen comprises a polysaccharide-protein conjugate. Optionally, one of the polysaccharide and/or the protein is a Group A Streptococcus antigen. For example, the protein may be a Streptococcus pyogenes antigen like a SpyCEP polypeptide, a SpyAD polypeptide and/or a SLO polypeptide. Alternatively or additionally, the polysaccharide may be a Streptococcus pyogenes antigen such as GAC.

### Polysaccharide-Protein Conjugate

The term “polysaccharide” refers to any linear or branched polymer consisting of monosaccharide residues, usually linked by glycosidic linkages, and thus includes oligosaccharides. By “protein” we mean or include any linear or branched molecule consisting of amino acid residues.

The term “polysaccharide-protein conjugate” refers to a molecule formed by a covalent linkage between a protein and a polysaccharide.

GAC (the Lancefield group A carbohydrate) is a polysaccharide comprising a polyrhamnose backbone with N-acetylglucosamine (GlcNAc) side chains, and may comprise the repeating units [→3)α-Rha(1→2) [β-GlcNAc(1→3)]α-Rha(1→] (referred to herein as a “GAC subunit”). GAC is the main component of the GAS cell wall.

The term “GAC” refers to a GAC polysaccharide derived from a strain of Streptococcus pyogenes. Alternatively, the term “GAC” may refer to a fragment or subunit (repeating unit) of the GAC polysaccharide such as one or more GAC subunits. Optionally, GAC comprises 18 GAC subunits.

By a fragment of a polysaccharide we mean polysaccharides that are truncated compared to the wild-type polysaccharide (e.g., have an average [mean] number of monosaccharide units that is smaller compared to the wild-type polysaccharide). Polysaccharide truncation can be achieved by any suitable means known in the art such as chemical digestion, in vitro polysaccharide synthesis of polysaccharide with fewer monosaccharide units than wild-type, or genetic modification of polysaccharide producing strains.

By a variant of a polysaccharide we mean or include that one or more chemical group of the polysaccharide backbone and/or side chain(s) is modified compared to wild-type polysaccharide. Polysaccharide modification can be achieved by any suitable means known in the art such as chemical reaction or genetic modification of polysaccharide producing strains.

Polysaccharide antigens such as GAC may be excellent antigens for stimulating B cell mediated immunity. However, it may be advantageous to conjugate GAC to a protein such as a carrier protein. Suitable proteins may include tetanus toxoid, diphtheria toxoid, CRM₁₉₇ or a protein from Streptococcus pyogenes such as a SpyAD polypeptide, a SpyCEP polypeptide and/or a SLO polypeptide.

Optionally, the carrier protein (i.e. the protein in the polysaccharide-protein conjugate) is CRM₁₉₇. CRM₁₉₇ is a non-toxic mutant of diphtheria toxin which has a single mutation at position 52 substituting glutamic acid for glycine. The mutation at position 52 causes loss of the ADP-ribosyltransferase activity of native diphtheria toxin. The polypeptide sequence of CRM₁₉₇ is provided in SEQ ID NO: 7.

The polypeptide may be conjugated to the protein using any suitable conjugation method.

Attachment of the polysaccharide to the protein is preferably via a —NH2 group, e.g., through the side chain(s) of a lysine residue(s) or arginine residue(s) in the carrier polypeptide. Where the polysaccharide has a free aldehyde group, this group can react with an amine in the protein to form a conjugate by reductive amination. Attachment to the carrier may also be via a —SH group, e.g., through the side chain(s) of a cysteine residue(s) in the carrier polypeptide. Alternatively, the polysaccharide may be attached to the carrier protein via a linker molecule.

The polysaccharide will typically be activated or functionalised prior to conjugation. Activation may involve, for example, cyanylating reagents such as CDAP (1-cyano-4-dimethylamino pyridinium tetrafluoro borate). Other suitable techniques use carbodiimides, hydrazides, active esters, norborane, p-nitrobenzoic acid, N-hydroxysuccinimide, S-NHS, EDC, TSTU (see, e.g., the introduction to WO 98/42721).

Direct linkages to the protein may comprise oxidation of the polysaccharide followed by reductive amination with the protein, as described in, for example, U.S. Pat. Nos. 4,761,283 and 4,356,170. Linkages via a linker group may be made using any known procedure, for example, the procedures described in U.S. Pat. Nos. 4,882,317 and 4,695,624. Typically, the linker is attached via an anomeric carbon of the polysaccharide. A preferred type of linkage is an adipic acid linker, which may be formed by coupling a free-NH2 group (e.g., introduced to a polysaccharide by amination) with adipic acid (using, for example, diimide activation), and then coupling a protein to the resulting saccharide-adipic acid intermediate (see, e.g., EP-B-0477508, Mol. Immunol, (1985) 22, 907-919, and EP-A-0208375). A similar preferred type of linkage is a glutaric acid linker, which may be formed by coupling a free-NH group with glutaric acid in the same way. Adipic and glutaric acid linkers may also be formed by direct coupling to the polysaccharide, i.e., without prior introduction of a free group, e.g., a free-NH group, to the polysaccharide, followed by coupling a protein to the resulting saccharide-adipic/glutaric acid intermediate. Another preferred type of linkage is a carbonyl linker, which may be formed by reaction of a free hydroxyl group of a modified polysaccharide with CDI (Bethell G. S. et al. (1979) J. Biol. Chem. 254, 2572-4 and Hearn M. T. W. (1981) J. Chromatogr. 218, 509-18); followed by reaction with a protein to form a carbamate linkage. Other linkers include β-propionamido (WO00/10599), nitrophenyl-ethylamine (Gever et al. (1979) Med. Microbiol. Immunol. 165, 171-288), haloacyl halides (U.S. Pat. No. 4,057,685), glycosidic linkages (U.S. Pat. Nos. 4,673,574; 4,761,283; and 4,808,700), 6-aminocaproic acid (U.S. Pat. No. 4,459,286), N-succinimidyl-3-(2-pyridyldithio)-propionate (SPDP)(U.S. Pat. No. 5,204,098), adipic acid dihydrazide (ADH)(U.S. Pat. No. 4,965,338), C4 to C12 moieties (U.S. Pat. No. 4,663,160), etc. Carbodiimide condensation can also be used (WO2007/000343).

A bifunctional linker may be used to provide a first group for coupling to an amine group in the polysaccharide (e.g., introduced to the polysaccharide by amination) and a second group for coupling to the carrier (typically for coupling to an amine in the carrier). Alternatively, the first group is capable of direct coupling to the polysaccharide, i.e., without prior introduction of a group, e.g., an amine group, to the polysaccharide.

Alternatively, the polysaccharide may be conjugated to the protein by a method in which the polysaccharide is exposed to random oxidation followed by reacting the oxidised polysaccharide with sodium cyanoborohydride, as disclosed in WO2022/101434 or in the Examples herein.

### SpyCEP Polypeptide

The at least one Group A Streptococcus antigen may comprise a SpyCEP polypeptide. Similarly, the protein in the polysaccharide-protein conjugate may comprise a SpyCEP polypeptide.

The term “SpyCEP polypeptide” refers to SpyCEP or a fragment or variant thereof. Suitable SpyCEP polypeptides comprise or consist of SEQ ID NO: 1 or 2 or a fragment or variant thereof. Another suitable SpyCEP polypeptide is the SpyCEP polypeptide of NCBI reference sequence WP_010921938.1.

The SpyCEP polypeptide may not comprise aspartate at a position corresponding to position 151 and/or serine at a position corresponding to position 617. References to amino acids at a position corresponding to “position 151 and “position 617” refer to amino acids at a position corresponding to the 151^{st} or 617^{th} amino acid in SEQ ID NO: 1 or SEQ ID NO: 2. Optionally, the SpyCEP polypeptide comprises alanine at a position corresponding to position 151 and/or alanine at a position corresponding to position 617. Mutations at positions corresponding to positions 151 and 617 may reduce the proteolytic activity of the SpyCEP polypeptide, making it safer to administer as part of a vaccine. Optionally, the SpyCEP polypeptide has reduced proteolytic activity against interleukin 8 (IL-8) relative to a wild type SpyCEP polypeptide. Optionally, the SpyCEP polypeptide has 50% or less proteolytic activity against interleukin 8 (IL-8) relative to a wild type SpyCEP polypeptide. A suitable assay to determine proteolytic activity against interleukin 8 is set out in the section entitled “Assay to determine proteolytic activity against interleukin 8”.

The SpyCEP polypeptide may comprise:
(i) an amino acid sequence that is at least 90%, at least 95%, at least 98%, at least 99%, or 100% identical to a contiguous fragment of at least 1000, at least 1200, at least 1400, at least 1500, or at least 1550 amino acids of SEQ ID NO: 1 or 2; or
(ii) an amino acid sequence that is at least 90%, at least 95%, at least 98%, at least 99%, or 100% identical to SEQ ID NO: 1 or 2; or
(iii) an amino acid sequence of SEQ ID NO: 1 or 2.

The SpyCEP polypeptide is preferably immunogenic. For example, administration of the SpyCEP polypeptide into mice may raise antibodies. The SpyCEP polypeptide may raise at least 50% (for example at least 80% or at least 95%) of the number of antibodies when administered to mice compared to a SpyCEP polypeptide of SEQ ID NO: 1 or SEQ ID NO: 2. Suitable assays for determining the number of antibodies raised are set out in the section entitled “Immunogenicity assays”.

The SpyCEP polypeptide may comprise an amino acid sequence that is at least 98%, at least 99%, or 100% identical to a contiguous fragment of at least 1400, at least 1500, or at least 1550 amino acids of SEQ ID NO: 1 or 2. The SpyCEP polypeptide may comprise an amino acid sequence that is at least 98%, at least 99%, or 100% identical to SEQ ID NO: 1 or 2.

### SpyAD Polypeptide

The at least one Group A Streptococcus antigen may comprise a SpyAD polypeptide. Similarly, the protein in the polysaccharide-protein conjugate may comprise a SpyAD polypeptide.

The term “SpyAD polypeptide” refers to SpyAD or a fragment or variant thereof. Suitable SpyAD polypeptides comprise or consist of SEQ ID NO: 3 or 4 or a fragment or variant thereof. Another suitable SpyAD polypeptide is the SpyAD polypeptide of NCBI reference sequence WP_010921884.1.

The SpyAD polypeptide may comprise:
(i) an amino acid sequence that is at least 90%, at least 95%, at least 98%, at least 99%, or 100% identical to a contiguous fragment of at least 600, at least 650, at least 700, at least 750, at least 770, or at least 800 amino acids of SEQ ID NO: 3 or 4; or
(ii) an amino acid sequence that is at least 90%, at least 95%, at least 98%, at least 99%, or 100% identical to SEQ ID NO: 3 or 4; or
(iii) an amino acid sequence of SEQ ID NO: 3 or 4.

The SpyAD polypeptide is preferably immunogenic. For example, administration of the SpyAD polypeptide into mice may raise antibodies. The SpyAD polypeptide may raise at least 50% (for example at least 80% or at least 95%) of the number of antibodies when administered to mice compared to a SpyAD polypeptide of SEQ ID NO: 3 or SEQ ID NO:

4. Suitable assays for determining the number of antibodies raised are set out in the section entitled “Immunogenicity assays”.

The SpyAD polypeptide may comprise an amino acid sequence that is at least 98%, at least 99%, or 100% identical to a contiguous fragment of at least 750, at least 770, or at least 800 amino acids of SEQ ID NO: 3 or 4. The SpyCEP polypeptide may comprise an amino acid sequence that is at least 98%, at least 99%, or 100% identical to SEQ ID NO: 3 or 4.

### SLO Polypeptide

The at least one Group A Streptococcus antigen may comprise a SLO (streptolysin O) polypeptide. Similarly, the protein in the polysaccharide-protein conjugate may comprise a SLO polypeptide.

The term “SLO polypeptide” or “streptolysin O polypeptide” refers to streptolysin O or a fragment or variant thereof. Suitable SLO polypeptides comprise or consist of SEQ ID NO: 5 or 6 or a fragment or variant thereof. Another suitable SLO polypeptide is the SLO polypeptide of NCBI reference sequence WP_010921831.1.

The SLO polypeptide may not comprise proline at a position corresponding to position 427 and/or tryptophan at a position corresponding to position 535. References to amino acids at a position corresponding to “position 427 and “position 535” refer to amino acids at a position corresponding to the 427th or 535th amino acid in SEQ ID NO: 1 or SEQ ID NO: 2. Optionally, the SLO polypeptide comprises leucine at a position corresponding to position 427 and/or phenylalanine at a position corresponding to position 535. Mutations at positions corresponding to positions 427 and 535 may reduce the hemolytic activity of the SLO polypeptide, making it safer to administer as part of a vaccine. Optionally, the SLO polypeptide has 50% or less hemolytic activity relative to a wild type SpyCEP polypeptide. A suitable assay to determine proteolytic activity against interleukin 8 is set out in the section entitled “Assay to determine hemolytic activity”.

The SLO polypeptide may comprise:
(i) an amino acid sequence that is at least 90%, at least 95%, at least 98%, at least 99%, or 100% identical to a contiguous fragment of at least 350, at least 400, at least 450, at least 500, or at least 530 amino acids of SEQ ID NO: 5 or 6; or
(ii) an amino acid sequence that is at least 90%, at least 95%, at least 98%, at least 99%, or 100% identical to SEQ ID NO: 5 or 6; or
(iii) an amino acid sequence of SEQ ID NO: 5 or 6.

The SLO polypeptide is preferably immunogenic. For example, administration of the SLO polypeptide into mice may raise antibodies. The SLO polypeptide may raise at least 50% (for example at least 80% or at least 95%) of the number of antibodies when administered to mice compared to a SLO polypeptide of SEQ ID NO: 5 or SEQ ID NO: 6. Suitable assays for determining the number of antibodies raised are set out in the section entitled “Immunogenicity assays”.

The SLO polypeptide may comprise an amino acid sequence that is at least 98%, at least 99%, or 100% identical to a contiguous fragment of at least 1400, at least 1500, or at least 1550 amino acids of SEQ ID NO: 5 or 6. The SLO polypeptide may comprise an amino acid sequence that is at least 98%, at least 99%, or 100% identical to SEQ ID NO: 5 or 6.

TLR7 Agonist and/or Benzonapthyridine Compound

The immunogenic composition of the invention comprises a TLR7 agonist and/or a benzonapthyridine compound. Methods of the invention are methods of making immunogenic compositions that comprise a TLR7 agonist and/or a benzonapthyridine compound.

A TLR7 agonist is a compound that is capable of activating TLR7. Typically TLR7 agonists are small molecules. The skilled person is aware of a number of different TLR7 agonists. Examples include LHD153R, imiquimod, resiquimod, NKTR-262, RG-7854, DSP-0509, BDB-001, BDC-1001, LJC-165, SHR-2150, JNJ-4964, vesatolimod, RO-7119929, BNT-411, APR-003. The skilled person is also able to determine whether a given molecule is acting as a TLR agonist. A suitable assay for determining whether a compound is a TLR7 agonist is provided in Russo et al.; Blood; 117:5683-5691. For example, the TLR7 agonist activity of a compound may be assessed by culturing pDCs with IL-3, stimulating the pDCs with the compound and measuring cytokine concentrations in the supernatant. Detection of cytokines and IFN-α in the supernatant is a marker of TLR7 agonist activity.

A TLR7 agonist may also be an agonist of another TLR such as TLR8. However, in some embodiments the TLR7 agonists preferentially activate TLR7 and do not activate other TLRs.

The TLR7 agonist may comprise or consist of a benzonapthyridine compound or a pharmaceutically acceptable salt thereof. The TLR7 agonist may be LHD153 and/or LHD153R. The TLR7 agonist may be 3-(5-amino-2-(2-methyl-4-(2-(2-(2-phosphonoethoxy)ethoxy)ethoxy)phenethyl)benzo[f][1,7]naphthyridin-8-yl)propanoic acid, or a pharmaceutically acceptable salt thereof.

Optionally, the benzonapthyridine compound is a compound which comprises a benzonapthyridine moiety, as shown below.

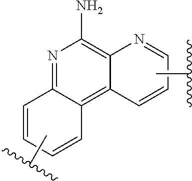

Typically the benzonapthyridine compound is a compound of formula (I). Methods of manufacturing suitable benzonapthyridine compounds of formula (I) are disclosed in WO2011/027222 and WO2013/131985.

Optionally, the benzonapthyridine compound comprises or consists of a compound of Formula (I),

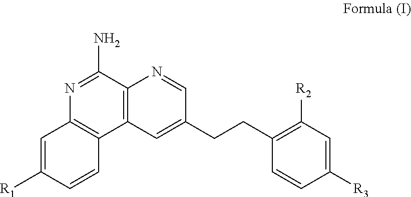

wherein:
R¹ is H, C1-C6alkyl, —C(R⁵)₂OH, —L¹R⁵, —L¹R⁶, —L²R⁵, —L²R⁶, —OL²R⁵, or —OL²R⁶;
L¹ is —C(O)— or —O—;
L² is C1-C6alkylene, C2-C6alkenylene, arylene, heteroarylene or —((CR⁴R⁴)ₚO)_{q}(CH₂)ₚ— wherein the C1-C6alkylene and C2-C6alkenylene of L² are optionally substituted with ₁ to 4 fluoro groups;
each L³ is independently selected from C1-C6alkylene and —((CR⁴R⁴)ₚO)_{q}(CH₂)ₚ— wherein the C1-C6alkylene of L³ is optionally substituted with 1 to 4 fluoro groups;
L⁴ is arylene or heteroarylene;
R² is H or C1-C6alkyl;
R³ is selected from C1-C4alkyl, —L³R⁵, —L¹R⁵, —L³R⁷, —L³L⁴L³R⁷, —L³L⁴R⁵, —L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵, and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁰;
R⁷ is —CF₂P(O)(OR⁹)₂ or —C(O)OR¹⁰;
R⁸ is H or C1-C4alkyl;
each R⁹ is independently selected from H and C1-C6alkyl;
R¹⁰ is H or C1-C4alkyl;
each p is independently selected from 1, 2, 3, 4, 5 and 6, and
q is 1, 2, 3 or 4;
   or a pharmaceutically acceptable salt thereof.

Suitable benzonapthyridine compounds include:
4-(4-(2-(5-amino-8-methylbenzo[f][1,7]naphthyridin-2-yl)ethyl)-3-methylphenoxy)-1,1-difluorobutylphosphonic acid;
3-(5-amino-2-(4-(4,4-difluoro-4-phosphonobutoxy)-2-methylphenethyl)benzo[f][1,7]naphthyridin-8-yl)propanoic acid;
3-(5-amino-2-(4-(2-(3, 3-difluoro-3-phosphonopropoxy)ethoxy)-2-methylphenethyl)benzo[f][1,7]naphthyridin-8-yl)propanoic acid;
3-(5-amino-2-(2-methyl-4-(2-(2-(2-phosphonoethoxy)ethoxy)ethoxy)phenethyl)benzo[f][1,7] naphthyridin-8-yl)propanoic acid;
4-(2-(5-amino-8-methylbenzo[f][1,7]naphthyridin-2-yl)ethyl)-3-methylphenyl dihydrogen phosphate;
(4-(2-(5-amino-8-methylbenzo[f][1,7]naphthyridin-2-yl)ethyl)-3-methylphenoxy)methylphosphonic acid; 5-(4-(2-(5-amino-8-methylbenzo[f][1,7]naphthyridin-2-yl)ethyl)-3-methylphenoxy)-1,1-difluoropentylphosphonic acid;
4-(4-(2-(5-amino-8-methylbenzo[f][1,7]naphthyridin-2-yl)ethyl)-3-methylphenoxy)-1,1-difluorobutylphosphonic acid;
3-(2-(4-(2-(5-amino-8-methylbenzo[f][1,7]naphthyridin-2-yl)ethyl)-3-methylphenoxy) ethoxy)-1,1-difluoropropylphosphonic acid;
2-(4-((4-(2-(5-amino-8-methylbenzo[f][1,7]naphthyridin-2-yl)ethyl)-3-methylphenoxy)methyl)phenyl)-1,1-difluoroethylphosphonic acid;
2-(5-amino-2-(4-methoxy-2-methylphenethyl)benzo[f][1,7]naphthyridin-8-yl)-1,1-difluoro-2-oxoethylphosphonic acid;
(E)-2-(5-amino-2-(4-methoxy-2-methylphenethyl)benzo[f][1,7]naphthyridin-8-yl)vinylphosphonic acid;
2-(5-amino-2-(4-methoxy-2-methylphenethyl)benzo[f][1,7]naphthyridin-8-yl)ethylphosphonic acid;
(E)-2-(5-amino-2-(4-methoxy-2-methylphenethyl)benzo[f][1,7]naphthyridin-8-yl)-1-fluorovinylphosphonic acid;
3-((4-(2-(5-amino-8-methylbenzo[f][1,7]naphthyridin-2-yl)ethyl)-3-methylphenoxy)methyl)phenylphosphonic acid;
5-amino-2-(4-methoxy-2-methylphenethyl)benzo[f][1,7]naphthyridine-8-carbonylphosphonic acid;
3-(5-amino-2-(2-methyl-4-(3-phosphonopropoxy)phenethyl)benzo[f][1,7]naphthyridin-8-yl)propanoic acid;
(4-(2-(5-amino-8-methylbenzo[f][1,7]naphthyridin-2-yl)ethyl)-3-methylphenyl)(hydroxy)methylenediphosphonic acid;
3-(5-amino-2-(4-(2-(2-(3,3-difluoro-3-phosphonopropoxy)ethoxy)ethoxy)-2-methylphenethyl)benzo[f][1,7]naphthyridin-8-yl)propanoic acid;
(5-amino-2-(4-methoxy-2-methylphenethyl)benzo[f][1,7]naphthyridin-8-yl)(hydroxy)methylenediphosphonic acid;
3-(5-amino-2-(2-methyl-4-(2-(2-phosphonoethoxy)ethoxy)phenethyl)benzo[f][1,7] naphthyridin-8-yl)propanoic acid;
2-(4-(2-(5-amino-8-methylbenzo[f][1,7]naphthyridin-2-yl)ethyl)-3-methylphenoxy)ethylphosphonic acid;
6-(4-(2-(5-amino-8-methylbenzo[f][1,7]naphthyridin-2-yl)ethyl)-3-methylphenoxy) hexylphosphonic acid;
6-(4-(2-(5-amino-8-methylbenzo[f][1,7]naphthyridin-2-yl)ethyl)-3-methylphenoxy)-1,1-difluorohexylphosphonic acid;
4-((4-(2-(5-amino-8-methylbenzo[f][1,7]naphthyridin-2-yl)ethyl)-3-methylphenoxy)methyl)benzylphosphonic acid;
2-(2-(2-(4-(2-(5-amino-8-methylbenzo[f][1,7]naphthyridin-2-yl)ethyl)-3-methylphenoxy) ethoxy)ethoxy)ethylphosphonic acid;
3-[5-amino-2-(2-[4-[2-(3,3-difluoro-3-phosphonopropoxy) ethoxy]-2-methylphenyl]ethyl)benzo[f]1,7-naphthyridin-8-yl]propanoic acid;
[5-[4-(2-{5-amino-8-methylbenzo[f]1,7-naphthyridin-2-yl)ethyl)-3-methylphenoxy] pentyl)phosphonic acid;
(4-[4-(2-(5-amino-8-methylbenzo[f]1,7-naphthyridin-2-yl}ethyl)-3-methylphenoxy]butyl}phosphonic acid
   and pharmaceutically acceptable salts thereof.

Optionally, the benzonapthyridine compound is 3-(5-amino-2-(2-methyl-4-(2-(2-(2-phosphonoethoxy)ethoxy)ethoxy)phenethyl)benzo[f][1,7]naphthyridin-8-yl)propanoic acid, or a pharmaceutically acceptable salt thereof.

Optionally, the benzonapthyridine compound comprises or consists of an arginine salt, (for example, a D-arginine salt or an L-arginine salt, preferably an L-arginine salt), of the compound of formula (I). Optionally, the benzonapthyridine compound is an arginine salt of 3-(5-amino-2-(2-methyl-4-(2-(2-(2-phosphonoethoxy)ethoxy)ethoxy)phenethyl)benzo[f][1,7]naphthyridin-8-yl)propanoic acid. Optionally, the benzonapthyridine compound is an L-arginine salt of 3-(5-amino-2-(2-methyl-4-(2-(2-(2-phosphonoethoxy)ethoxy)ethoxy)phenethyl)benzo[f][1,7]naphthyridin-8-yl)propanoic acid.

An arginine salt of the compound of formula (I), wherein:
R¹ is —(CH₂)₂C(O)OH;
R² is C1 alkyl; and
R³ is —O(((CH₂)₂)O)₂ (CH₂)₂—P(O)(OH)₂
   may also be referred to as LHD153R.

### Aluminium Salt

The immunogenic composition of the invention comprises an aluminium salt. Methods of the invention are methods of making immunogenic compositions that comprise an aluminium salt.

The aluminium salt may act as an adjuvant and/or an absorbent. An “adjuvant” generally refers to a substance that increase the immune response to an antigen. An “absorbent” generally refers to a solid substrate to which other components of the immunogenic composition, such as the at least one antigen, at least one Group A Streptococcus antigen, the TLR7 agonist and/or the benzonapthyridine compound may bind, attach or adsorb (for example, by Van der Waals interactions or hydrogen bonding). Particular aluminium salts include aluminium hydroxide (such as ALHYDROGEL®), aluminium phosphate, aluminium hydroxyphosphate, potassium aluminium sulphate and alum.

Optionally, the aluminium salt comprises or consists of aluminium phosphate and/or aluminium hydroxide. Optionally, the aluminium salt comprises or consists of aluminium phosphate.

### Mixtures of Components in the Immunogenic Composition

The at least one Group A Streptococcus antigen may comprise a polysaccharide-protein conjugate. In such embodiments, the ratio of the concentration (w/v) of the polysaccharide in the polysaccharide-protein conjugate to the concentration (w/v) of the TLR7 agonist or benzonapthyridine compound in the immunogenic composition may be between 1:0.01 and 1:50, between 1:0.1 and 1:20, between 1:0.1 and 1:10, between 1:0.5 and 1:10, between 0.75:1 and 1:5, between 1:1 and 1:3, between 1:1.5 and 1:2.5, or around 1:2. Optionally, the ratio of the concentration (w/v) of the polysaccharide in the polysaccharide-protein conjugate to the concentration (w/v) of the TLR7 agonist or benzonapthyridine compound in the immunogenic composition is between 1:1.5 and 1:2.5. Optionally, the ratio of the concentration (w/v) of the polysaccharide in the polysaccharide-protein conjugate to the concentration (w/v) of aluminium in the immunogenic composition is between 0.25:1 and 1:250, between 1:25 and 1:200, between 1:5 and 1:100, between 0.75:10 and 1:50, between 1:10 and 1:30, between 1:15 and 1:25, or around 1:20. Optionally, the ratio of the concentration (w/v) of the polysaccharide in the polysaccharide-protein conjugate to the concentration (w/v) of aluminium in the immunogenic composition is between 1:15 and 1:21.

The at least one Group A Streptococcus antigen may comprise a SpyCEP polypeptide. In such embodiments, the ratio of the concentration (w/v) of the SpyCEP polypeptide to the concentration (w/v) of the TLR7 agonist or benzonapthyridine compound in the immunogenic composition may be between 1:0.1 and 1:50, between 1:0.1 and 1:20, between 1:0.1 and 1:10, between 1:0.5 and 1:10, between 0.75:1 and 1:5, between 0.75:1 and 1:3, between 0.75.1 and 1:2.5, or around 1:1. Optionally wherein the ratio of the concentration (w/v) of the SpyCEP polypeptide to the concentration (w/v) of the TLR7 agonist or benzonapthyridine compound in the immunogenic composition is between 0.75:1 and 1:2.5. Optionally, the ratio of the concentration (w/v) of the SpyCEP polypeptide to the concentration (w/v) of aluminium in the immunogenic composition is between 0.25:1 and 1:250, between 1:25 and 1:200, between 1:5 and 1:100, between 0.75:10 and 1:50, between 0.75:10 and 1:30, between 1:5 and 1:15, or around 1:11. Optionally, the ratio of the concentration (w/v) of the SpyCEP polypeptide to the concentration (w/v) of aluminium in the immunogenic composition is between 1:5 and 1:15.

The at least one Group A Streptococcus antigen may comprise a SpyAD polypeptide. In such embodiments, the ratio of the concentration (w/v) of the SpyAD polypeptide to the concentration (w/v) of the TLR7 agonist or benzonapthyridine compound in the immunogenic composition may be between 1:0.1 and 1:50, between 1:0.1 and 1:20, between 1:0.1 and 1:10, between 1:0.5 and 1:10, between 0.75:1 and 1:5, between 0.75:1 and 1:3, between 0.75.1 and 1:2.5, or around 1:1. Optionally, the ratio of the concentration (w/v) of the SpyAD polypeptide to the concentration (w/v) of the TLR7 agonist or benzonapthyridine compound in the immunogenic composition is between 0.75:1 and 1:2.5. Optionally, the ratio of the concentration (w/v) of the SpyAD polypeptide to the concentration (w/v) of aluminium in the immunogenic composition is between 0.25:1 and 1:250, between 1:25 and 1:200, between 1:5 and 1:100, between 0.75:10 and 1:50, between 0.75:10 and 1:30, between 1:5 and 1:15, or around 1:11. Optionally, the ratio of the concentration (w/v) of the SpyAD polypeptide to the concentration (w/v) of aluminium in the immunogenic composition is between 1:5 and 1:15.

The at least one Group A Streptococcus antigen may comprise a SLO polypeptide. In such embodiments, the ratio of the concentration (w/v) of the SLO polypeptide to the concentration (w/v) of the TLR7 agonist or benzonapthyridine compound in the immunogenic composition may be between 1:0.1 and 1:50, between 1:0.1 and 1:20, between 1:0.1 and 1:10, between 1:0.5 and 1:10, between 0.75:1 and 1:5, between 0.75:1 and 1:3, between 0.75.1 and 1:2.5, or around 1:1. Optionally, the ratio of the concentration (w/v) of the SLO polypeptide to the concentration (w/v) of the TLR7 agonist or benzonapthyridine compound in the immunogenic composition is between 0.75:1 and 1:2.5. Optionally, the ratio of the concentration (w/v) of the SLO polypeptide to the concentration (w/v) of aluminium in the immunogenic composition is between 0.25:1 and 1:250, between 1:25 and 1:200, between 1:5 and 1:100, between 0.75:10 and 1:50, between 0.75:10 and 1:30, between 1:5 and 1:15, or around 1:11. Optionally, the ratio of the concentration (w/v) of the SLO polypeptide to the concentration (w/v) of aluminium in the immunogenic composition is between 1:5 and 1:15.

Optionally, the immunogenic composition:
(i) comprises a polysaccharide-protein conjugate, wherein the polysaccharide is GAC and the protein is CRM₁₉₇, a SpyAD polypeptide, a SpyCEP polypeptide and a SLO polypeptide; and
(ii) wherein the SpyAD polypeptide is at least 98% identical to SEQ ID NO: 4, the SpyCEP polypeptide is at least 98% identical to SEQ ID NO: 2, and the SLO polypeptide is at least 98% identical to SEQ ID NO: 6.

Optionally, the immunogenic composition:
(i) comprises a polysaccharide-protein conjugate, wherein the polysaccharide is GAC and the protein is CRM₁₉₇, a SpyAD polypeptide, a SpyCEP polypeptide and a SLO polypeptide; and
(ii) wherein the SpyAD polypeptide is at least 98% identical to SEQ ID NO: 3, the SpyCEP polypeptide is at least 98% identical to SEQ ID NO: 1, and the SLO polypeptide is at least 98% identical to SEQ ID NO: 5.

In embodiments in which the immunogenic composition comprises a polysaccharide-protein conjugate, a SpyAD polypeptide, a SpyCEP polypeptide and a SLO polypeptide, the immunogenic composition may comprise these components at the following ratios:
the ratio of the concentration (w/v) of the polysaccharide in the polysaccharide-protein conjugate to the concentration (w/v) of the TLR7 agonist or benzonapthyridine compound in the immunogenic composition is between 0.75:1 and 1:5;
the ratio of the concentration (w/v) of the polysaccharide in the polysaccharide-protein conjugate to the concentration (w/v) of aluminium in the immunogenic composition is between 0.75:10 and 1:50;
the ratio of the concentration (w/v) of the SpyCEP polypeptide to the concentration (w/v) of the TLR7 agonist or benzonapthyridine compound in the immunogenic composition is between 0.75:0.5 and 1:2.5;
the ratio of the concentration (w/v) of the SpyCEP polypeptide to the concentration (w/v) of aluminium in the immunogenic composition is between 1:5 and 1:25;
the ratio of the concentration (w/v) of the SpyAD polypeptide to the concentration (w/v) of the TLR7 agonist or benzonapthyridine compound in the immunogenic composition is between 0.75:0.5 and 1:2.5;
the ratio of the concentration (w/v) of the SpyAD polypeptide to the concentration (w/v) of aluminium in the immunogenic composition is between 1:3 and 1:25;
the ratio of the concentration (w/v) of the SLO polypeptide to the concentration (w/v) of the TLR7 agonist or benzonapthyridine compound in the immunogenic composition is between 0.75:0.5 and 1:2.5; and
the ratio of the concentration (w/v) of the SLO polypeptide to the concentration (w/v) of aluminium in the immunogenic composition is between 1:3 and 1:25.

Optionally, the immunogenic composition comprises between 0.5 mg/ml and 10 mg/ml, between 0.5 mg/ml and 5 mg/ml, or around 1 mg/ml of the aluminium salt. Optionally, the immunogenic composition comprises between 0.01 mg/ml and 5 mg/ml, between 0.05 mg/ml and 2 mg/ml, or around 0.1 mg/ml of the TLR7 agonist and/or the benzonapthyridine compound. Optionally, the at least one Group A Streptococcus antigen comprises a polysaccharide-protein conjugate comprising GAC (optionally GAC conjugated to CRM₁₉₇), and the immunogenic composition comprises between 5 μg and 75 μg, between 10 μg and 50 μg, or around 25 μg of polysaccharide-protein conjugate Optionally, the at least one Group A Streptococcus antigen comprises a SpyAD polypeptide and the immunogenic composition comprises between 10 μg and 150 μg, between 25 μg and 75 μg, or around 50 μg of the SpyCEP polypeptide. Optionally, the at least one Group A Streptococcus antigen comprises a SpyCEP polypeptide and the immunogenic composition comprises between 10 μg and 150 μg, between 25 μg and 75 μg, or around 50 μg of the SpyCEP polypeptide. Optionally, the at least one Group A Streptococcus antigen comprises a SLO polypeptide and the immunogenic composition comprises between 10 μg and 150 μg, between 25 μg and 75 μg, or around 50 μg of the SLO polypeptide.

Optionally, the immunogenic composition comprises:
between 0.5 mg/ml and 10 mg/ml of the aluminium salt;
between 0.01 mg/ml and 5 mg/ml of the TLR7 agonist and/or the benzonapthyridine compound;
between 5 μg and 75 μg of a polysaccharide-protein conjugate comprising GAC (optionally GAC conjugated to CRM₁₉₇);
between 5 μg and 75 μg of a SpyAD polypeptide;
between 5 μg and 75 μg of a SpyCEP polypeptide; and
between 5 μg and 75 μg of a SLO polypeptide.

Optionally, at least 50%, at least 75%, or at least 80% of the at least one antigen or the at least one Group A streptococcus antigen in the immunogenic composition is adsorbed to the aluminium salt. Optionally, at least 50%, at least 75%, or at least 80% of the TLR7 agonist and/or benzonapthyridine compound is adsorbed to the aluminium salt. The level of adsorption of a test compound such as a Group A streptococcal antigen and/or a TLR agonists and/or a benzonapthyridine compound to an aluminium salt may be measured by mixing the aluminium salt with the test compound, centrifuging the mixture at 14,000×g for 2-5 minutes and then measuring the concentration of the test compound in the supernatant (for example using absorbance at 280 nm to calculate the concentration of a protein test compound).

### Immunogenicity of the Components of the Immunogenic Compositions

The immunogenic composition may raise a greater number of antibodies when administered to mice compared to an equivalent immunogenic composition that does not comprise the TLR7 agonist and/or benzonapthyridine compound. Optionally, the immunogenic composition raises a greater number of antibodies when administered to mice at a dose of 5000 ng compared to an equivalent immunogenic composition that does not comprise the TLR7 agonist and/or benzonapthyridine compound. Optionally, the immunogenic composition raises a greater number of antibodies when administered to mice at a dose of 5000 ng compared to an equivalent immunogenic composition that does not comprise the TLR7 agonist and/or benzonapthyridine compound when measured using serum collected 27 days after administration.

By the phrase “equivalent immunogenic composition that does not comprise the TLR7 agonist and/or benzonapthyridine compound” we mean an immunogenic composition which is identical to the immunogenic composition except that it lacks the TLR7 agonist and/or the benzonapthyridine compound. By administered to mice “at a dose of 5000 ng” is meant that each/any protein antigen(s)(such as a SpyCEP polypeptide, a SpyAD polypeptide and/or a SLO polypeptide) in the immunogenic composition is administered at a dose of 5000 ng and each/any polysaccharide conjugate antigen (such as GAC-CRM₁₉₇) is administered at a dose of 2500 ng. The number of antibodies raised when administered to mice may be determined using an assay described under the heading “immunogenicity assays”.

Optionally, the immunogenic composition raises a greater number of antibodies against at least one of the following antigens:
(i) SpyCEP;
(ii) SpyAD;
(iii) SLO; and/or
(iv) GAC,
   when administered to mice compared to an equivalent immunogenic composition that does not comprise the TLR7 agonist and/or benzonapthyridine compound. For example, if the at least one Group A Streptococcus antigen comprises a SpyCEP polypeptide, the immunogenic composition may raise a greater number of antibodies against SpyCEP when administered to mice compared to an equivalent immunogenic composition that does not comprise the TLR7 agonist and/or benzonapthyridine compound. Similarly, if the at least one Group A Streptococcus antigen comprises a SpyAD polypeptide, the immunogenic composition may raise a greater number of antibodies against SpyAD when administered to mice compared to an equivalent immunogenic composition that does not comprise the TLR7 agonist and/or benzonapthyridine compound. Similarly, if the at least one Group A Streptococcus antigen comprises a SLO polypeptide, the immunogenic composition may raise a greater number of antibodies against SLO when administered to mice compared to an equivalent immunogenic composition that does not comprise the TLR7 agonist and/or benzonapthyridine compound. Optionally, when the immunogenic composition comprises a SpyCEP polypeptide, the SpyCEP polypeptide that is administered to mice is administered at a dose of 5000 ng. Optionally, when the immunogenic composition comprises a SpyAD polypeptide, the SpyAD polypeptide that is administered to mice is administered at a dose of 5000 ng. Optionally, when the immunogenic composition comprises a SLO polypeptide, the SLO polypeptide that is administered to mice is administered at a dose of 5000 ng.

Optionally, the immunogenic composition raises a greater number of antibodies against at least one of the following antigens:
(i) SpyCEP;
(ii) SpyAD;
(iii) SLO; and/or
(iv) GAC,
   when administered to mice at a 5000 ng dose compared to an equivalent immunogenic composition that does not comprise the TLR7 agonist and/or benzonapthyridine compound.

Optionally, the immunogenic composition raises a greater number of antibodies against all of the following antigens SpyCEP, SpyAD, SLO and GAC when administered to mice compared to an equivalent immunogenic composition that does not comprise the TLR7 agonist and/or benzonapthyridine compound. Optionally, the immunogenic composition raises a greater number of antibodies against all of the following antigens SpyCEP, SpyAD, SLO and GAC when administered to mice at a 5000 ng dose compared to an equivalent immunogenic composition that does not comprise the TLR7 agonist and/or benzonapthyridine compound.

Optionally, the immunogenic composition raises a greater number of antibodies against SpyCEP when administered to mice at a 5000 ng dose (optionally when the number of antibodies is measured using serum collected 27 days after administration) compared to an equivalent immunogenic composition that does not comprise the TLR7 agonist and/or benzonapthyridine compound. Optionally, the immunogenic composition raises at least 2 times or at least 5 times the number of antibodies against SpyCEP when administered to mice at a 5000 ng dose (optionally when the number of antibodies is measured using serum collected 27 days after administration) compared to an equivalent immunogenic composition that does not comprise the TLR7 agonist and/or benzonapthyridine compound.

Optionally, the immunogenic composition raises a greater number of antibodies against SpyAD when administered to mice at a 5000 ng dose (optionally when the number of antibodies is measured using serum collected 27 days after administration) compared to an equivalent immunogenic composition that does not comprise the TLR7 agonist and/or benzonapthyridine compound. Optionally, the immunogenic composition raises at least 2 times the number of antibodies against SpyAD when administered to mice at a 5000 ng dose (optionally when the number of antibodies is measured using serum collected 27 days after administration) compared to an equivalent immunogenic composition that does not comprise the TLR7 agonist and/or benzonapthyridine compound.

Optionally, the immunogenic composition raises a greater number of antibodies against SLO when administered to mice at a 5000 ng dose (optionally when the number of antibodies is measured using serum collected 27 days after administration) compared to an equivalent immunogenic composition that does not comprise the TLR7 agonist and/or benzonapthyridine compound. Optionally, the immunogenic composition raises at least 2 times or at least 5 times the number of antibodies against SLO when administered to mice at a 5000 ng dose (optionally when the number of antibodies is measured using serum collected 27 days after administration) compared to an equivalent immunogenic composition that does not comprise the TLR7 agonist and/or benzonapthyridine compound.

Optionally, the immunogenic composition raises a greater number of antibodies against GAC-CRM₁₉₇ when administered to mice at a 5000 ng dose (which correlates to 2500 ng of GAC-CRM₁₉₇ as explained in the definition of 5000 ng dose given above)(optionally when the number of antibodies is measured using serum collected 27 days after administration) compared to an equivalent immunogenic composition that does not comprise the TLR7 agonist and/or benzonapthyridine compound. Optionally, the immunogenic composition raises at least 2 times or at least 5 times the number of antibodies against GAC-CRM₁₉₇ when administered to mice at a 5000 ng dose (optionally when the number of antibodies is measured using serum collected 27 days after administration) compared to an equivalent immunogenic composition that does not comprise the TLR7 agonist and/or benzonapthyridine compound.

Optionally, when administered to mice at a 5000 ng dose, the immunogenic composition raises at least 2 times the number of antibodies against SpyAD, SpyCEP, SLO and GAC-CRM₁₉₇ compared to an equivalent immunogenic composition that does not comprise the TLR7 agonist and/or benzonapthyridine compound when measured using serum collected 27 days after administration.

The immunogenic composition may raise a greater number of functional antibodies when administered to mice compared to an equivalent immunogenic composition that does not comprise the TLR7 agonist and/or benzonapthyridine compound. Optionally, the immunogenic composition raises a greater number of functional antibodies when administered to mice at a dose of 5000 ng compared to an equivalent immunogenic composition that does not comprise the TLR7 agonist and/or benzonapthyridine compound. Optionally, the immunogenic composition raises a greater number of functional antibodies when administered to mice at a dose of 5000 ng compared to an equivalent immunogenic composition that does not comprise the TLR7 agonist and/or benzonapthyridine compound when measured using serum collected 27 days after administration. The number of functional antibodies raised may be determined using the assays described under the headings “Assay to determine proteolytic activity against interleukin 8” and “Assay to determine hemolytic activity”.

Optionally, the immunogenic composition raises a greater number of functional antibodies against SpyCEP and SLO when administered to mice compared to an equivalent immunogenic composition that does not comprise the TLR7 agonist and/or benzonapthyridine compound. For example, if the at least one Group A Streptococcus antigen comprises a SpyCEP polypeptide, the immunogenic composition may raise a greater number of functional antibodies against SpyCEP when administered to mice compared to an equivalent immunogenic composition that does not comprise the TLR7 agonist and/or benzonapthyridine compound. Similarly, if the at least one Group A Streptococcus antigen comprises a SLO polypeptide, the immunogenic composition may raise a greater number of functional antibodies against SLO when administered to mice compared to an equivalent immunogenic composition that does not comprise the TLR7 agonist and/or benzonapthyridine compound.

Optionally, the immunogenic composition raises a greater number of functional antibodies against SpyCEP when administered to mice at a 5000 ng dose (optionally when the number of antibodies is measured using serum collected 42 days after administration wherein a booster dose of the immunogenic composition was administered at day 28) compared to an equivalent immunogenic composition that does not comprise the TLR7 agonist and/or benzonapthyridine compound. Optionally, the immunogenic composition raises at least 2 times, at least 5 times or at least 7.5 times the number of functional antibodies against SpyCEP when administered to mice at a 5000 ng dose (optionally when the number of antibodies is measured using serum collected 42 days after administration wherein a booster dose of the immunogenic composition was administered at day 28) compared to an equivalent immunogenic composition that does not comprise the TLR7 agonist and/or benzonapthyridine compound.

Optionally, the immunogenic composition raises a greater number of functional antibodies against SLO when administered to mice at a 5000 ng dose (optionally when the number of antibodies is measured using serum collected 42 days after administration wherein a booster dose of the immunogenic composition was administered at day 28) compared to an equivalent immunogenic composition that does not comprise the TLR7 agonist and/or benzonapthyridine compound. Optionally, the immunogenic composition raises at least 2 times, or at least 5 times the number of functional antibodies against SLO when administered to mice at a 5000 ng dose (optionally when the number of antibodies is measured using serum collected 42 days after administration wherein a booster dose of the immunogenic composition was administered at day 28) compared to an equivalent immunogenic composition that does not comprise the TLR7 agonist and/or benzonapthyridine compound.

Optionally, when administered to mice at a 5000 ng dose, the immunogenic composition raises at least 5 times the number of functional antibodies against SpyCEP and SLO compared to an equivalent immunogenic composition that does not comprise the TLR7 agonist and/or benzonapthyridine compound when measured using serum collected 42 days after administration wherein a booster dose of the immunogenic composition was administered at day 28.

### Immunogenicity Assays

Immunogenicity levels (the number of antibodies raised against a given antigen such as SpyCEP, SpyAD, SLO or GAC) may be determined in one of several ways known to the skilled person.

For example, the skilled person may use an ELISA based method or may use a fluorescence immunoassay. When a fluorescence immunoassay is used, the level of antibodies can be measured in units of RLU/ml. Fluorescence beads may be used, where each bead is coupled to a unique bead region. The beads are associated with unique spots on the fluorescence spectrum. Different antigens may be coupled to the beads in the different regions. Suitable antigens include GAC, SpyCEP (for example a protein of SEQ ID NO: 1 or 2), SpyAD (for example a protein of SEQ ID NO: 3 or 4) and SLO (for example a protein of SEQ ID NO: 5 or 6). For example, beads from a first region may be coupled to SpyCEP antigen, and beads from a second region may be coupled to SpyAD antigen. Sera from mice immunised with the immunogenic composition of the invention may be applied to the beads and allowed to bind to the antigens coupled to the beads. The remaining serum may then be removed with a washing step, and a detection mixture comprising biotinylated detection antibodies (that bind to mice antibodies) and a streptavidin-phycoerythrin (PE) reporter applied. The beads can be excited one by one to determine the bead region and corresponding antigen. A second laser then determines that magnitude of the PE-derived signal, which is proportional to the amount of analyte bound. The user can, thereby, determining the number of bound antibodies associated with each antigen.

A suitable fluorescence immunoassay uses the Luminex (® machine. A suitable fluorescence (Luminex®) assay is described in Example 2.

To determine whether a test immunogenic composition raises greater numbers of antibodies when administrated to mice compared to a reference immunogenic composition (for example an equivalent immunogenic composition that does not comprise a TLR7 agonist and/or benzonapthyridine compound), the level of antibodies raised by the test immunogenic composition may be measured using one of the assays above, and the level of antibodies raised by the reference immunogenic composition should be measured using an identical assay, and the two antibody levels compared.

### Assay to Determine Proteolytic Activity Against Interleukin 8

The number of functional antibodies against SpyCEP (anti-SpyCEP antibodies) may be measured using an IL-8 cleavage assay. Wild type SpyCEP has a proteolytic activity that allows it to cleave IL-8, and so the ability of antibodies raised against SpyCEP to prevent IL-8 cleavage by SpyCEP is a measure of how functional the antibodies are.

A suitable IL-8 cleavage assay may involve pre-incubating wild-type SpyCEP with serum from mice immunised with serial dilutions of the immunogenic composition to be tested or a buffer only control. Human IL-8 can then be added and the reaction incubated at 37 °C for 2 hours. The reaction mixture may then be diluted 20-fold and incubated with monoclonal antibodies directed against IL-8 to determine the level of IL-8 remaining (uncleaved). The level of IL-8 remaining should be compared with the level of IL-8 remaining in a control experiment (an equivalent experiment except that the serum is from mice prior to administration of the immunogenic composition to be tested). The IC50 may be calculated by determining the serum concentration that achieved 50% inhibition of hemolysis (50% less reduction in IL-8 compared to the control experiment).

A suitable IL-8 cleavage assay is described in Example 2.

To determine whether a test immunogenic composition raises greater numbers of functional anti-SpyCEP antibodies when administered to mice compared to a reference immunogenic composition (for example an equivalent immunogenic composition that does not comprise a TLR7 agonist and/or benzonapthyridine compound), the level of functional anti-SpyCEP antibodies raised by the test immunogenic composition may be measured using one of the assays above, and the level of functional anti-SpyCEP antibodies raised by the reference immunogenic composition should be measured using an identical assay, and the two functional anti-SpyCEP antibody levels compared.

### Assay to Determine Hemolytic Activity

The number of functional antibodies raised against SLO (anti-SLO antibodies) may be measured using a hemolysis assay. Wild type SLO is haemolytic, and so the ability of antibodies raised against SLO to prevent the haemolytic activity by SLO is a measure of how functional the antibodies are.

A suitable hemolysis assay may involve preparing a red blood cell suspension (e.g. rabbit red blood cells). In parallel, one may prepare mixtures of a serial dilution of serum from mice immunised with the immunogenic composition to be tested and pre-incubate them with a fixed concentration of wild type SLO toxin to form a test pre-incubated mixture. The rabbit blood cells may then be mixed with the pre-incubated mixture and the incubated for 30 minutes at 37 °C. The blood cells may then be centrifuged for 5 minutes at 1000×g, and the level of hemolysis can be measured by looking at how much haemoglobin is released. The level of haemoglobin released may be measured by reading absorbance at 540 nm. Th level of haemoglobin released should be compared to the level of haemoglobin released in a control experiment (an equivalent experiment except that the serum is from mice prior to administration of the immunogenic composition to be tested). The serum titre corresponding to the dilution factor which provides 50% inhibition of cleavage (i.e. 50% of the haemoglobin released compared to the control experiment), is determined.

A suitable hemolysis assay is described in Example 2.

To determine whether a test immunogenic composition raises greater numbers of functional anti-SLO antibodies when administered to mice compared to a reference immunogenic composition (for example an equivalent immunogenic composition that does not comprise a TLR7 agonist and/or benzonapthyridine compound), the level of functional anti-SLO antibodies raised by the test immunogenic composition may be measured using one of the assays above, and the level of functional anti-SLO antibodies raised by the reference immunogenic composition should be measured using an identical assay, and the two functional anti-SLO antibody levels compared.

### Pharmaceutically Acceptable Excipients

The immunogenic composition may further comprise a pharmaceutically acceptable excipient.

Typical ‘pharmaceutically acceptable excipients’ include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. Pharmaceutically acceptable excipients may also contain diluents, such as water, saline, glycerol, etc. Additionally, auxiliary substances, such as wetting or emulsifying agents, pH buffering substances, and the like, may be present. Sterile pyrogen-free, Tris-buffered physiologic saline is a suitable carrier particularly when using aluminium adjuvants since the phosphate in phosphate buffered saline may interfere with outer membrane vesicle binding to aluminium.

Typical pharmaceutical excipients may include one of more of Tris buffer, histidine, sodium chloride and sodium phosphate. Optionally, the immunogenic composition comprises tris buffer, histidine, sodium chloride and sodium phosphate. Optionally, the immunogenic composition comprises tris buffer at a concentration between 0.5 mM and 25 mM, between 1 mM and 10 mM, or around 2 mM. Optionally, the immunogenic composition comprises histidine at a concentration between 1 mM and 50 mM, between 5 mM and 25 mM, or around 10 mM. Optionally, the immunogenic composition comprises sodium chloride at a concentration between 50 mM and 300 mM, between 75 mM and 250 mM, or around 129 mM. Optionally, the immunogenic composition comprises sodium phosphate at a concentration between 1 mM and 25 mM, between 2 mM and 10 mM, or around 4 mM.

Thus, immunogenic compositions of the invention may be useful as vaccines. Vaccines according to the invention may either be prophylactic (i.e. to prevent infection) or therapeutic (i.e. to treat infection), but will typically be prophylactic.

Immunogenic compositions used as vaccines comprise an immunologically effective amount of antigen(s), as well as any other components, as needed. By “immunologically effective amount”, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Immunogenic compositions of the invention may include an antimicrobial, particularly when packaged in multiple dose formats.

### Methods of Treatment

The present invention also provides the immunogenic composition or the vaccine of the invention for use in a method of preventing infection by Group A Streptococcus. The present invention provides a method of preventing infection and/or autoimmune disease following infection by Group A Streptococcus comprising administering an effective amount of the immunogenic composition or vaccine of the invention. The present invention also provides a use of the immunogenic composition or the vaccine of the invention for preparing a medicament for use in a method of preventing infection and/or autoimmune disease following infection by Group A Streptococcus.

The term “treating” includes both therapeutic treatment and prophylactic or preventative treatment, wherein the object is to prevent or lessen infection. For example, treating may include directly affecting or curing, suppressing, inhibiting, preventing, reducing the severity of, delaying the onset of, reducing symptoms associated with, for example, infection, or a combination thereof. “Preventing” may refer, inter alia, to delaying the onset of symptoms, preventing relapse of a disease, and the like. “Treating” may also include “suppressing” or “inhibiting” an infection or illness, for example reducing severity, number, incidence or latency of symptoms, ameliorating symptoms, reducing secondary symptoms, reducing secondary infections, prolonging patient survival, or combinations thereof.

The term “preventing or treating Group A Streptococcus infection” in the method/immunogenic composition for use/use of the immunogenic composition in the manufacture of a medicament of the invention comprises raising an immune response in a subject. The immune response may be protective and may raises antibodies, such as IgG antibodies.

The subject of the invention is a mammal, optionally a human. Where the vaccine is for prophylactic use, the human may be an adult i.e. subject is 18 years old or above 18 years old. Where the vaccine is for prophylactic use, the human may be a child i.e. below 18 years old. Where the vaccine is for prophylactic use, the child may be between 12 to 72 months, preferably between 24 to 59 months, more preferably between 6 to 12 months.

Where the vaccine is for prophylactic use, the child may be around 9 months.

Where the vaccine is for therapeutic use, the human is preferably a child.

A vaccine intended for children may also be administered to adults e.g. to assess safety, dosage, or immunogenicity.

### Methods of Making the Immunogenic Composition

The invention further provides a method of making an immunogenic composition comprising at least one antigen, an aluminium salt and (a) a TLR7 agonist, and/or (b) a benzonapthyridine compound comprising preparing a mixture of:
(i) the at least one antigen pre-absorbed on the aluminium salt and the TLR7 agonist and/or benzonapthyridine compound;
(ii) the at least one antigen and the TLR7 agonist and/or benzonapthyridine compound pre-absorbed on the aluminium salt; or
(iii) the at least one antigen pre-absorbed on the aluminium salt and the TLR7 agonist and/or benzonapthyridine compound pre-absorbed on the aluminium salt.

The mixtures may be prepared by any suitable method. For example, the mixtures may be prepared by adding two components together (like the alum and the at least one antigen) and vortexing them or simply flicking them to mix them together. The mixtures may be prepared in any suitable mixing vessel. For example, the mixtures may be prepared in bulk in large vats for use in later syringe filling, or may be prepared directly in syringes ready to use to inject a patient.

The present invention further provides an immunogenic composition that is obtained or obtainable from the methods of making an immunogenic composition of the invention.

| Sequence ID | Nature of sequence |
|---|---|
| 1 | Double mutant SpyCEP mature polypeptide sequence |
| 2 | Full length native SpyCEP |
| 3 | SpyAD mature polypeptide sequence |
| 4 | SpyAD polypeptide sequence comprising N-terminal |
| | exclusion domain |
| 5 | Double mutant SLO mature polypeptide sequence |
| 6 | Full length native SLO |
| 7 | Mature CRM197 |

### EXAMPLES

### Example 1—Preparation of Vaccine Antigens

Vaccines are prepared containing four separate antigens. The first antigen was a conjugate of GAC and CRM₁₉₇, the second antigen is an SpyCEP polypeptide of SEQ ID NO: 1, the third antigen is a SpyAD polypeptide of SEQ ID NO: 3 and the fourth antigen is an SLO polypeptide of SEQ ID NO: 5.

### SpyAD

The upstream process begins with thawing one MCB GMP seed (E. coli BL21 (DE3) pET24 strain expressing the SpyAD antigen), followed by inoculum preparation (25 °C, 200 rpm, 0.7<OD<2.0) and then the fermentation process (fermentation medium contains yeast extract, 30 °C, pH 7.2, pO₂ set point 30%). The recombinant protein SpyAD expression is triggered by addition of isopropyl-β-D-thiogalactoside (IPTG) when the culture reaches an OD value of 6.0+1.0 (induction phase). The process ends 4 hours after induction. At the end of the fermentation, the broth is harvested and centrifuged (12,227 g×30 min). The pellet obtained is collected and stored at −70 °C.

The recombinant protein SpyAD is extracted from biomass with a homogenizer (Avestin, 10 Ksi, 2 cycles), then the lysate is clarified by centrifugation (12,227 g×30 min) and orthogonal filtration (Sartorius, Sartobran 300). The subsequent purification consists of three chromatographic steps:
Capto Q (Cytiva, Buffer A: 30 mM Tris-HCl 90 mM NaCl, pH 9; Buffer B: 30 mM Tris-HCl 150 mM NaCl, pH 9, step elution),
Butyl Sepharose 4 μF hs (Cytiva, Buffer A: 450 mM (NH₄)₂SO₄, 10 mM KPi, pH 7.2; Buffer B: 200 mM (NH₄)₂SO₄, 10 mM KPi, pH 7.2; step elution),
Ceramic Hydroxyapatite (CHT)(Type I-40 μm, Biorad, Buffer A: 10 mM NaPi pH 7.2; Buffer B: 200 mM NaPi, pH 7.2; gradient elution).

A first TFF (tangential flow filtration) step (30 KDa cut-off, TMP 1.1 barg, DP 0.2 barg) between Butyl and CHT steps is required to exchange buffer and put the SpyAD intermediate in the CHT loading buffer. A second TFF step (30 KDa cut-off, TMP 1.1 barg, DP 0.2 barg) after the CHT step is needed to put the purified SpyAD antigen in the formulation buffer (10 mM KPi, pH 7.2) and reach the target drug substance (DS) concentration. The Pre-Bulk is then filtered through a 0.2 μm membrane (Sartorius, Sartobran 150) to generate the final Bulk.

### SLO

The upstream process begins with thawing one MCB GMP seed (E. coli BL21 (DE3) pET24 strain expressing the SLO antigen), followed by inoculum preparation (25 °C, 200 rpm, 3<OD<7) and then the fermentation process (fermentation medium contains yeast extract, 25 °C, pH 7.2, pO₂ set point 30%). The recombinant protein SLO expression is triggered by addition of IPTG when the culture reaches an OD value of 5.0±2.0 (induction phase). The process ends 9 hours after induction. At the end of the fermentation, the broth is harvested and centrifuged (12,227 g×30 min). The pellet obtained is collected and stored at −70 °C.

The recombinant protein SLO is extracted from biomass with a homogenizer (Avestin, 10 Ksi, 2 cycles), then the lysate is clarified by centrifugation (12,227 g×30 min) and orthogonal filtration (Sartorius, Sartobran 300). The subsequent purification consists of three chromatographic steps:
Butyl Sepharose 4 μF hs (Cytiva, Buffer A: 2M NaCl, 20 mM NaPi, pH 7; Buffer B: 1.5M NaCl, 20 mM NaPi, pH 7; Buffer C: 20 mM NaPi, pH 7, step elution),
Capto Q (Cytiva, Buffer 20 mM NaPi pH 7, negative chromatography),
Ceramic Hydroxyapatite (Type I-40 μm, Biorad, Buffer A: 10 mM NaPi pH 6.8; Buffer B: 130 mM NaPi, pH 6.8; Buffer C: 250 mM NaPi, pH 6.8, step elution).

A final TFF step (30 KDa cut-off, TMP 1.0 barg, DP 1.0 barg) after the CHT step is needed to put the purified SLO antigen in the formulation buffer (10 mM KPi, pH 7.2) and reach the target DS concentration. The Pre-Bulk is then filtered through a 0.2 μm membrane (Sartorius, Sartobran 150) to generate the final Bulk.

### SpyCEP

The upstream process begins with thawing one MCB GMP seed (E. coli BL21 (DE3) pET24 strain expressing the SpyCEP antigen), followed by inoculum preparation (30 °C, 200 rpm, 1<OD<5) and then the fermentation process (fermentation medium contains yeast extract, 30 °C, pH 7.2, pO₂ set point 30%). The recombinant protein SpyCEP expression is triggered by addition of IPTG and decreasing temperature at 25 °C when the culture reaches an OD value ≥3.5 (induction phase). The process ends when the final culture's OD is 30±5. At the end of the fermentation, the broth is harvested and centrifuged (12,227 g×30 min). The pellet obtained is collected and stored at −70 °C.

The recombinant protein SpyCEP is extracted from biomass with a homogenizer (Avestin, 10 Ksi, 2 cycles), then the lysate is clarified by centrifugation (12,227 g×30 min) and orthogonal filtration (Sartorius, Sartobran 300). The subsequent purification consists of three chromatographic steps:
Capto Q (Cytiva, Buffer A: 10 mM NaPi pH 7.5; Buffer B: 10 mM NaPi, 0.2M NaCl pH 7.5, step elution),
Ceramic Hydroxyapatite (Type I-40 μm, Biorad, Buffer A: 10 mM NaPi pH 7.5; Buffer B: 80 mM NaPi, pH 7.5; step elution).
Phenyl Sepharose 6 μF hs (Cytiva, Buffer A: 2M NaCl, 10 mM NaPi, pH 7.5; Buffer B: 10 mM NaPi, pH 7.5; step elution),

A final TFF step (30KDa cut-off, TMP 0.8 barg, DP 0.5 barg) after the CHT step is needed to put the purified SpyCEP antigen in the formulation buffer (10 mM KPi, pH 7.2) and reach the target DS concentration. The Pre-Bulk is then filtered through a 0.2 μm membrane (Sartorius, Sartobran 150) to generate the final Bulk.

### GAC-rCRM

The upstream process begins with thawing two MCB GMP seeds (Streptococcus pyogenes emml knockout), followed by inoculum preparation and then the fermentation process. The fermentation medium contains Yeast Extract certified animal free. The Fermentation is stopped when the growth slows down consistently. At this point the OD value measured remains stable (or even at a lower value) compared with the previous sampling point. The final OD expected is 17±5. At the end of the fermentation, an inactivation phase will follow. This consists in heating the culture in the bioreactor at 100 °C for 2 hours. Afterwards, the culture is cooled down reaching 20 °C and the broth is harvested and centrifuged. The pellet obtained is collected and stored at −70 °C.

The GAC is extracted from biomass generating HNO₂ starting from NaNO₂ and H₃PO₄, at pH 3.2 at room temperature for 24 hours. The GAC is separated from lysate by centrifugation and clarified by orthogonal filtration. The subsequent purification consists of the following steps:
30K membrane TFF (TMP 0.7 barg, DP 0.6 barg)
Q Sepharose FF (Cytiva, Buffer 10 mM NaPi, 0.3M NaCl, pH 7.2, negative chromatography)
A further TFF step (30K membrane, TMP 0.7 barg, DP 0.6 barg) could be required to further concentrate the purified GAC, if the GAC concentration is lower than target concentration

The GAC intermediate is then filtered through a 0.2 μm membrane (hold point).

Then the GAC intermediate is activated with a solution of 200 mM NaIO₄ (8 mM final concentration) and left for 30 minutes in a climate chamber at 25 °C. Quenching is performed using a Na₂SO₃ solution (final concentration of 16 mM), mixing for 15-30 minutes at RT. The oxidized GAC is purified and exchanged in Borate buffer pH 8 with TFF 10K (TMP 0.5 barg, DP 1.0 barg) and centrifuge concentrators (3-10 KDa): the target GACox concentration is 130-140 mg/mL.

The rCRM (recombinant CRM₁₉₇) is concentrated up to 65-70 mg/mL and exchanged in 50 mM Borate buffer pH 8 before conjugation step with TFF and/or centrifuge concentrators (10 KDa membrane cut-off).

The conjugation step is done with a concentration of GACox and rCRM of 30 mg/mL with a ratio of 1:1 (w/w) for 24 hours at 37 °C. (mild mixing). The reaction starts with the addition of 200 mg/mL NaBH₃CN solution (final concentration 5 mg/mL). The reaction is stopped with 10 times dilution with PBX 1X and addition of a 20 mg/mL NaBH₄ solution with a ratio of 1:0.5=mg of GAC: mg of NaBH₄ for 2-4 hours at room temperature

The GAC-rCRM is purified and exchanged in PBS 1X by TFF 50K (TMP 0.3 barg, DP 0.5 barg), then filtered through a 0.2 μm membrane (Sartorius, Sartobran 150) to generate the final Bulk.

### Mixing

These four antigens were mixed with adjuvant in the concentrations set out in the following tables. The adjuvant used was either aluminium hydroxide alone, or a combination of aluminium hydroxide and LHD153R.

| | | | Formulation with | |
|---|---|---|---|---|
| Vaccine | Formulation with | | aluminium adjuvant and | |
| component | aluminium adjuvant | | LHD153R | |
| Alum (Al³⁺) | 1.05 | mg/ml | 1.05 | mg/ml |
| LHD153R | | | 0.1 | mg/ml |
| Tris | 2 | mM | 2 | mM |
| Histidine | 10 | mM | 10 | mM |
| Sodium | 154 | mM | 129.5 | mM |
| Chloride | | | | |
| Sodium | 6 | mM | 4 | mM |
| phosphate | | | | |

| | | |
|---|---|---|
| Antigens | High dose: 5000 ng SLO | High dose: 5000 ng SLO |
| | polypeptide, 5000 ng | polypeptide, 5000 ng |
| | SpyAD polypeptide, 5000 | SpyAD polypeptide, 5000 |
| | ng SpyCEP polypeptide, | ng SpyCEP polypeptide, |
| | 2500 ng GAC-CRM197 | 2500 ng GAC-CRM197 |
| | Medium dose: 625 ng SLO | Medium dose: 625 ng SLO |
| | polypeptide, 625 ng | polypeptide, 625 ng |
| | SpyAD polypeptide, 625 | SpyAD polypeptide, 625 |
| | ng SpyCEP polypeptide, | ng SpyCEP polypeptide, |
| | 313 ng GAC-CRM197 | 313 ng GAC-CRM197 |
| | Low dose: 78 ng SLO | Low dose: 78 ng SLO |
| | polypeptide, 78 ng SpyAD | polypeptide, 78 ng SpyAD |
| | polypeptide, 78 ng SpyCEP | polypeptide, 78 ng SpyCEP |
| | polypeptide, 39 ng GAC- | polypeptide, 39 ng GAC- |
| | CRM197 | CRM197 |

### Example 2—Comparing the Immunogenicity of the Formulations Comprising Alum or Alum and LHD153R in Mice

### Mice Immunisation Protocol

Groups of 8 CD1 mice were immunized with one of the 6 formulations set out above. The immunisations involved intraperitoneal immunization of 200 μl of each formulation at day 0 and day 28. Blood samples were collected at days 27 and 42, and the antibodies raised were studied using the following assays.

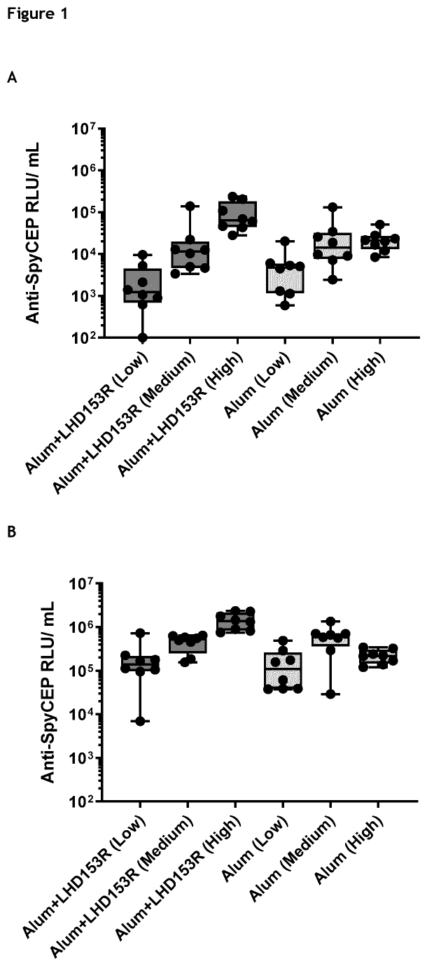

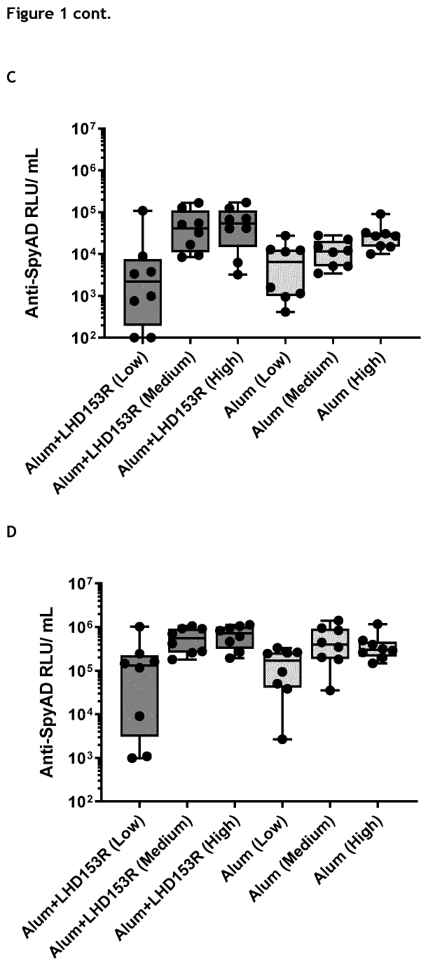

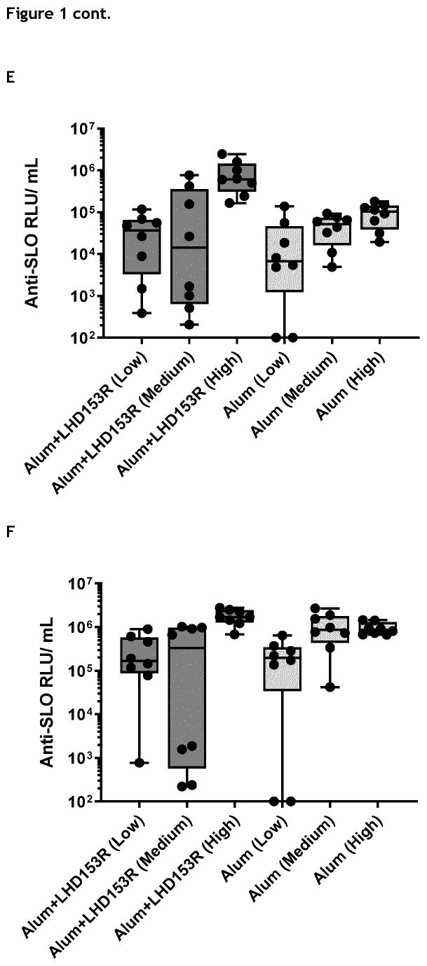

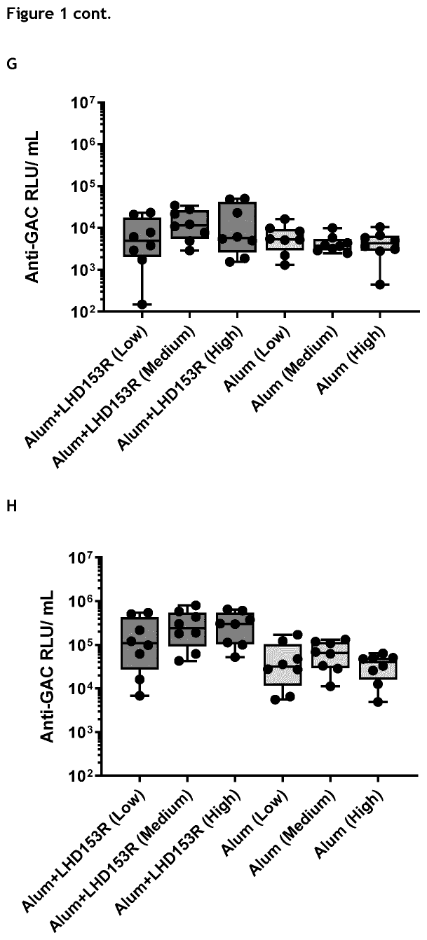

Assessment of level of anti-SLO, anti-SpyAD, anti-SpyCEP and anti-GAC antibody levels The levels of antibodies against SLO, SpyAD, SpyCEP and GAC in the blood samples taken from the mice immunized as described in the section entitled “Mice immunization protocol” was measured using the Luminex assay described in Keeley et al. Methods Protoc. 2022, 5, 55 but with the following modifications: anti-mouse or anti-rat II antibody PE were used and multiple sera dilutions have been tested (300 at the lowest, 3-fold apart for up to 7 dilutions) and the antibody concentration have been calculated as the median of all results falling within the dynamic range of the standard curve. The results of this assay are set out in FIG. 1.

### IL-8 Cleavage Inhibition Assay

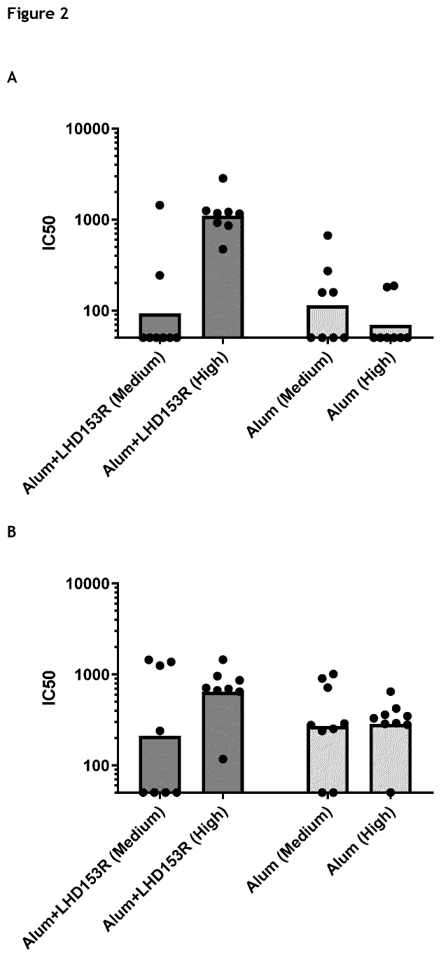

Post-second immunization individual sera isolated from the blood samples taken from the mice immunized as described in the section entitled “Mice immunization protocol” were tested in an IL-8 cleavage ELISA assay to evaluate their ability to block SpyCEP proteolytic activity. The assay was performed as previously described in Bensi et al. (Multi high-throughput approach for highly selective identification of vaccine candidates: the Group A Streptococcus case. Mol Cell Proteomics 2012, 11, (6), M111 015693.) with some modifications. Briefly, SpyCEP (5 ng/ml) was preincubated with mouse polyclonal anti-SpyCEP serum at four different dilutions (1:100, 1:300, 1:900, 1:2700) for 5 minutes at 4 °C in PBS 0.5 mg/ml BSA. Pre-incubation of SpyCEP with buffer only and with pre-immune serum were used as negative controls. Then, human IL-8 (Gibco, 10 ng/ml) was added and the reaction was incubated at 37 °C (reaction without enzyme was used as control). After 2 hours, each reaction mix was diluted 20-fold and incubated in 96-well plates coated with a blend of monoclonal antibodies directed against distinct epitopes of IL-8 (Life Technologies). The amount of IL-8 in each sample and in the control reaction (without the enzyme) was determined according to the manufacturer's protocols, using a standard curve of IL-8. Each serum dilution was tested twice, and the mean value with error bar was reported in the graph. Results are shown in panel A of FIG. 2 and expressed as the minimal sera dilution required to inhibit 50% of native SpyCEP activity (IC50) for each serum tested.

### In Vitro Hemolysis Assay

Pre-immune and post-second immunization individual sera isolated from the blood samples taken from the mice immunized as described in the section entitled “Mice immunization protocol” were tested in a hemolysis assay to evaluate their ability to block SLO hemolytic activity. The assay was performed as previously described in Bensi et al. with some modifications. Briefly, a red blood cell suspension was prepared by washing rabbit red blood cells (Emozoo) four times in PBS and resuspended in PBS (20% rabbit red blood cell suspension in PBS). Eight serial 2-fold dilutions of either anti-SLO sera or negative control pre-immune serum diluted in PBS with 0.5% BSA were prepared in 96 well round bottom plates then preincubated with 900 units/mL of SLO toxin (Sigma, diluted in PBS with 40 mM dithiothreitol, Invitrogen) at RT for 30 minutes (in a final volume of 150 μL). Following addition of rabbit blood cell suspension (50 μL), incubation was continued for 30 minutes at 37 °C. Plates were finally centrifuged for 5 minutes at 1000×g and the supernatant was carefully transferred to 96-well flat-bottomed plates. The absorbance of the released hemoglobin was read at 540 nm. Results are shown in panel B of FIG. 2 and expressed as the minimal sera dilution required to inhibit 50% of hemolysis (IC50) for each serum tested.

### Statistics

Mann-Whitney two-tailed test was used to compare the immune response elicited by two different antigens, Kruskal-Wallis test with Dunn's post hoc analysis was used for comparison among more than two groups. Wilcoxon test matched-pairs signed rank two-tailed test was performed to compare the response induced by the same antigen at day 27 vs day 42.

### Assessment of Antibody Type

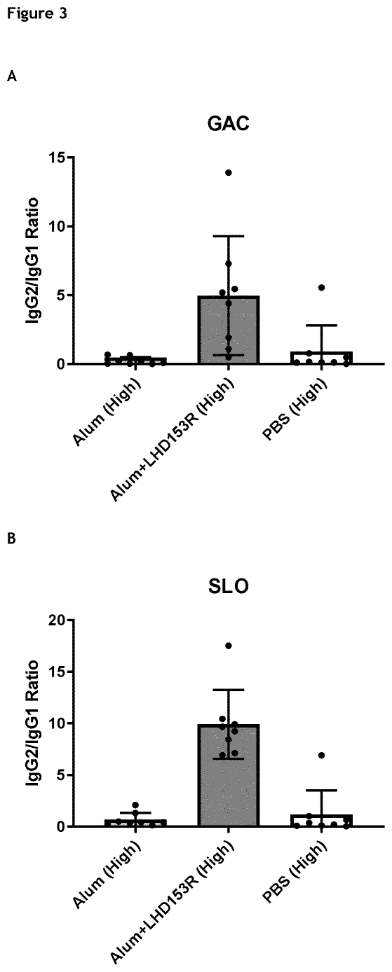

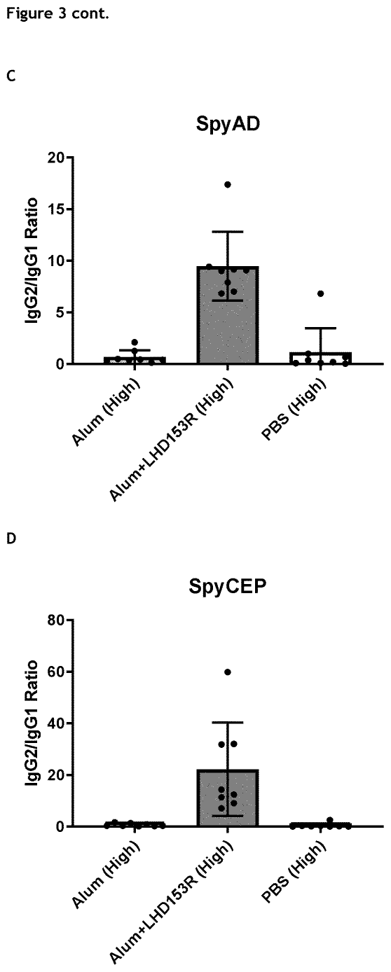

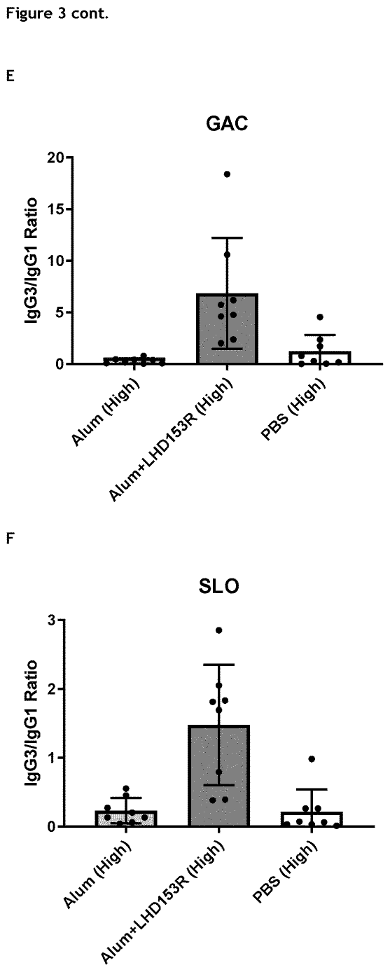

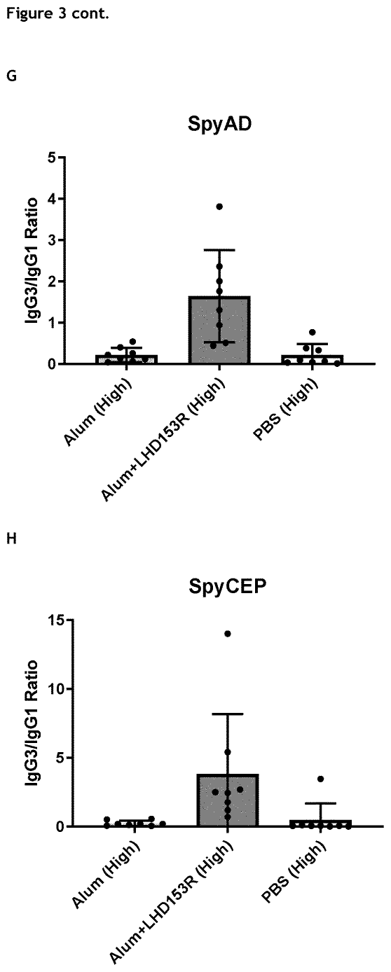

Post-second immunization individual sera isolated from the blood samples taken from the mice immunized as described in the section entitled “Mice immunization protocol” were tested to determine the isotype of the antibodies produced using a Luminex based assay working with the same principle as described under the heading “Assessment of level of anti-SLO, anti-SpyAD, anti-SpyCEP and anti-GAC antibody levels”. The assay was repeated in parallel to determine the RLU/mL on each individual sera at the highest vaccine dose tested using as secondary antibody antimouse-IgG1-PE, antimouse-IgG2a-PE, antimouse-IgG2b-PE, antimouse-IgG2c-PE and antimouse-IgG3-PE antibodies in standard assay. Results are shown in FIG. 3 and expressed as a ratio compared to IgG1 for each individual sera.

### Example 3—Comparing the Immunogenicity of the Formulations Comprising LHD153R+Alum or No Adjuvant in Mice

### Mice Immunisation Protocol

Groups of 8 CD1 mice were immunized with one of the six formulations described in the table below. The immunisations involved intraperitoneal immunization of 200 μl of each formulation at day 0 and day 28. Blood samples were collected at days 27 and 42, and the antibodies raised were studied using the following assays.

| | Formulation with | | | |
|---|---|---|---|---|
| Vaccine | aluminum adjuvant and | | Formulation with no | |
| component | LHD153R | | adjuvant | |
| Alum (Al³⁺) | 1.05 | mg/ml | NA | |
| LHD153R | 0.1 | mg/ml | NA | |
| Tris | 2 | mM | NA | |
| Histidine | 10 | mM | NA | |
| Sodium | 129.5 | mM | 154 | mM |
| Chloride | | | | |
| Sodium | 4 | mM | 10 | mM |
| phosphate | | | | |

| | | |
|---|---|---|
| Antigens | High dose: 5000 ng SLO | High dose: 5000 ng SLO |
| | polypeptide, 5000 ng | polypeptide, 5000 ng |
| | SpyAD polypeptide, 5000 | SpyAD polypeptide, 5000 |
| | ng SpyCEP polypeptide, | ng SpyCEP polypeptide, |
| | 2500 ng GAC-CRM197 | 2500 ng GAC-CRM197 |
| | Medium dose: 625 ng SLO | Medium dose: 625 ng SLO |
| | polypeptide, 625 ng | polypeptide, 625 ng |
| | SpyAD polypeptide, 625 | SpyAD polypeptide, 625 |
| | ng SpyCEP polypeptide, | ng SpyCEP polypeptide, |
| | 313 ng GAC-CRM197 | 313 ng GAC-CRM197 |
| | Low dose: 78 ng SLO | Low dose: 78 ng SLO |
| | polypeptide, 78 ng SpyAD | polypeptide, 78 ng SpyAD |
| | polypeptide, 78 ng SpyCEP | polypeptide, 78 ng SpyCEP |
| | polypeptide, 39 ng GAC- | polypeptide, 39 ng GAC- |
| | CRM197 | CRM197 |

### Assessment of Antibodies Produced

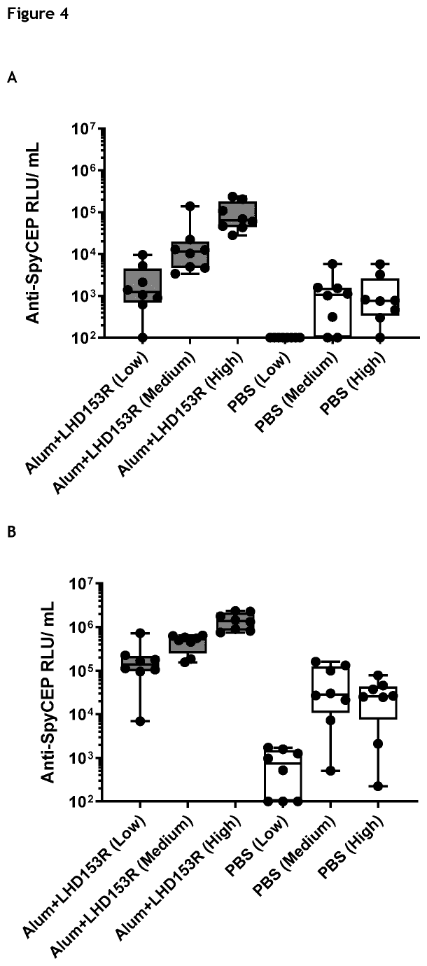

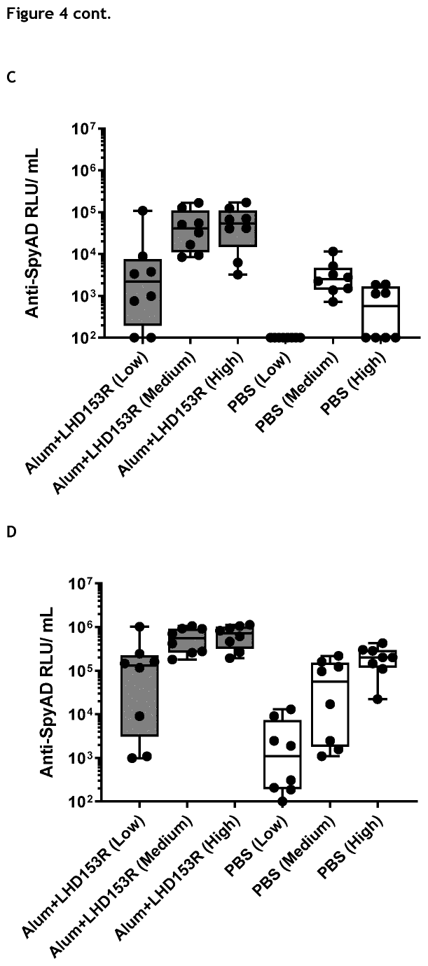

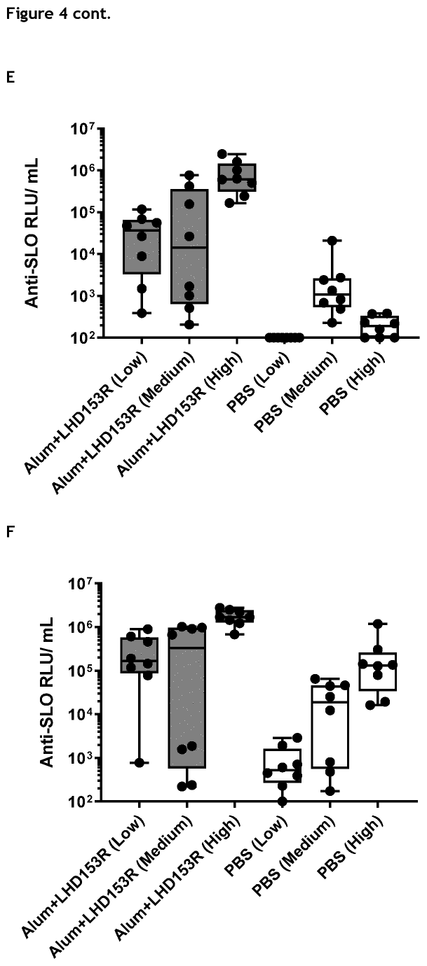

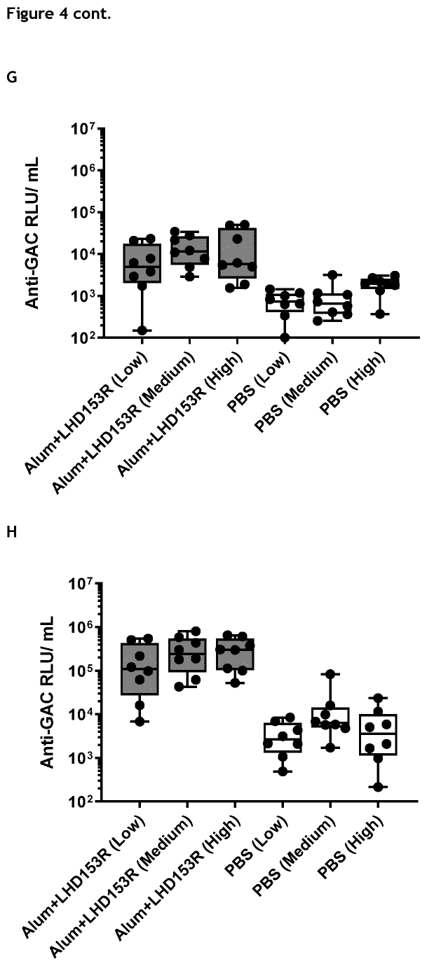

The levels of antibodies against SLO, SpyAD, SpyCEP and GAC in the blood samples taken from the mice immunized as described in the section entitled “Mice immunization protocol” was measured using the assay described under the heading “Assessment of level of anti-SLO, anti-SpyAD, anti-SpyCEP and anti-GAC antibody levels”. The results of this assay are set out in FIG. 4.

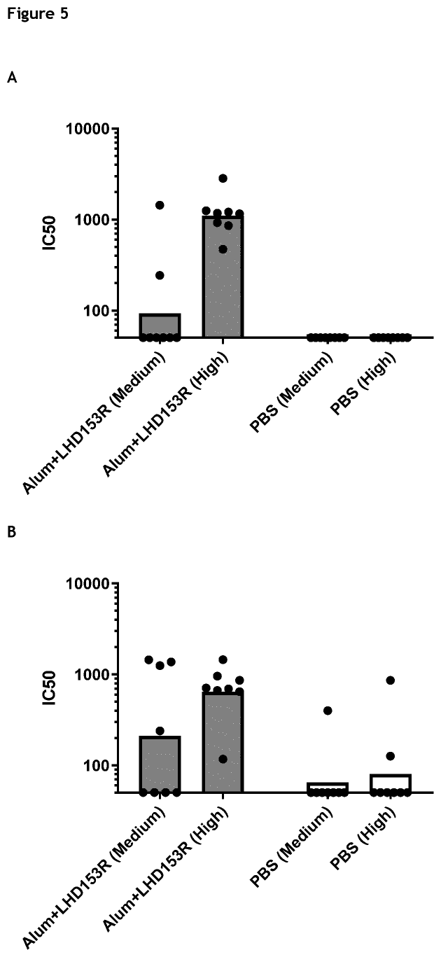

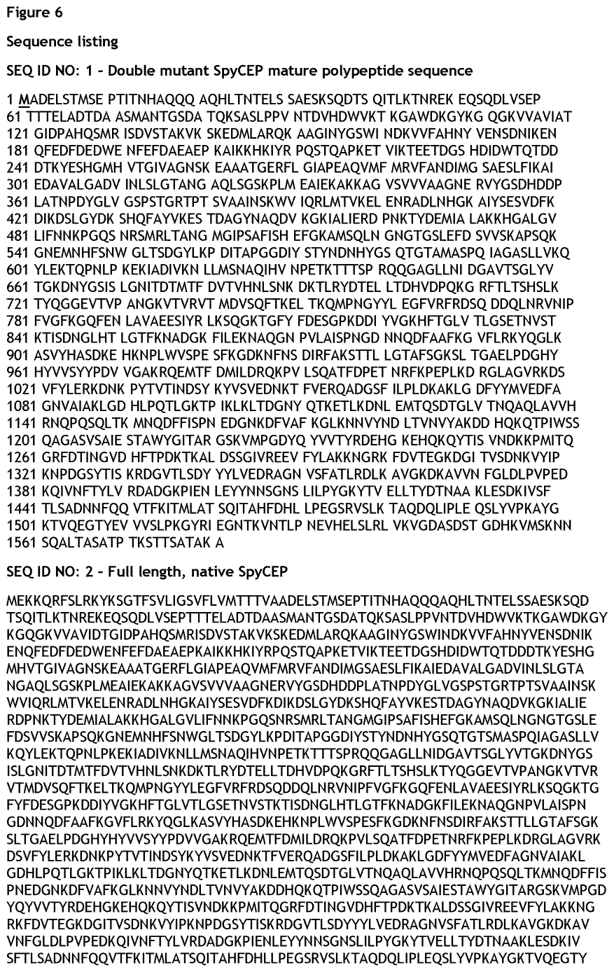

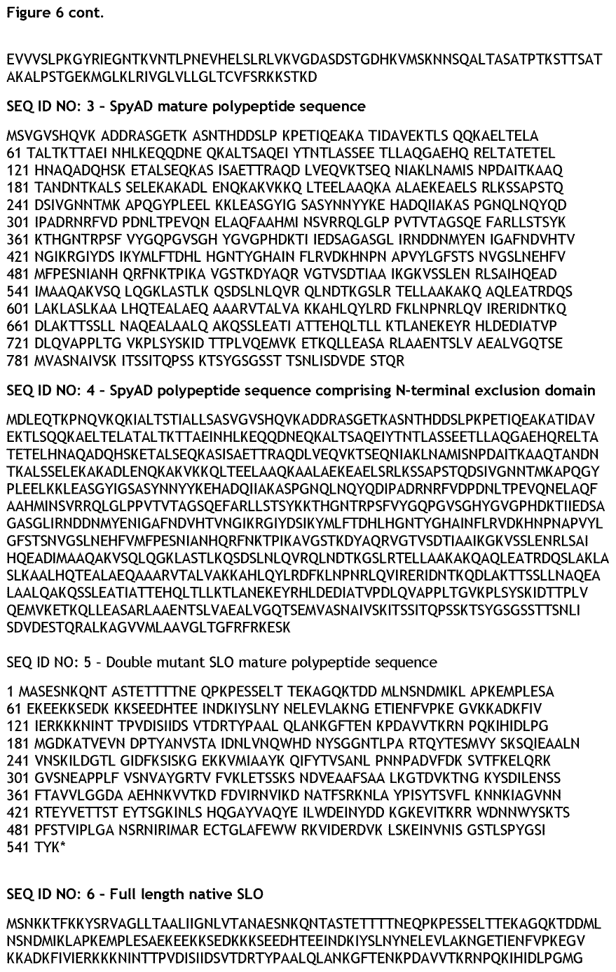

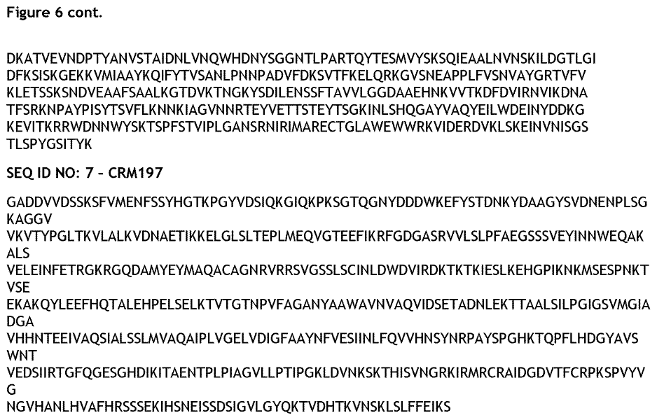

Pre-immune and post-second immunization individual sera isolated from the blood samples taken from the mice immunized as described in the section entitled “Mice immunization protocol” were tested for functional antibodies in an IL-8 cleavage ELISA assay and a hemolysis assay as described under the headings “IL-8 cleavage inhibition assay” and “In vitro hemolysis assay”. The results are set out in FIG. 5.

The invention described herein also relates to the following aspects:
1. An immunogenic composition comprising at least one Group A Streptococcus antigen, an aluminium salt and a TLR7 agonist.
2. An immunogenic composition comprising at least one Group A Streptococcus antigen, an aluminium salt and a benzonapthyridine compound.
3. A method of making an immunogenic composition comprising at least one antigen, an aluminium salt and (a) a TLR7 agonist, and/or (b) a benzonapthyridine compound comprising preparing a mixture of:
   (i) the at least one antigen pre-absorbed on the aluminium salt and the TLR7 agonist and/or benzonapthyridine compound;
   (ii) the at least one antigen and the TLR7 agonist and/or benzonapthyridine compound pre-absorbed on the aluminium salt; or
   (iii) the at least one antigen pre-absorbed on the aluminium salt and the TLR7 agonist and/or benzonapthyridine compound pre-absorbed on the aluminium salt.
4. An immunogenic composition that is obtained or obtainable from the method of aspect 3.
5. The immunogenic composition or method of aspect 3 or 4, wherein the at least one antigen comprises an antigen from a bacterium selected from the group consisting of Actinomyces (e.g., A. israelii), Bacillus (e.g., B. anthracis or B. cereus), Bartonella (e.g., B. henselae, or B. quintana), Bordetella (e.g., B. pertussis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. Typhi, S. Enteritidis, S. Paratyphi, S. Typhimurium, or S. Choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).
6. The immunogenic composition or method of any one of aspects 3 to 5, wherein the at least one antigen comprises an antigen from streptococcus.
7. The immunogenic composition or method of any one of the preceding aspects, wherein the aluminium salt comprises or consists of aluminium phosphate or aluminium hydroxide.
8. The immunogenic composition or method of any one of the preceding aspects, wherein the aluminium salt comprises or consists of aluminium hydroxide.
9. The immunogenic composition or method of any one of aspects 1, or 3 to 8, wherein the TLR7 agonist comprises or consists of a benzonapthyridine compound.
10. The immunogenic composition or method of any one of aspects 2 to 9, wherein the benzonapthyridine compound comprises or consists of a compound of Formula (I),

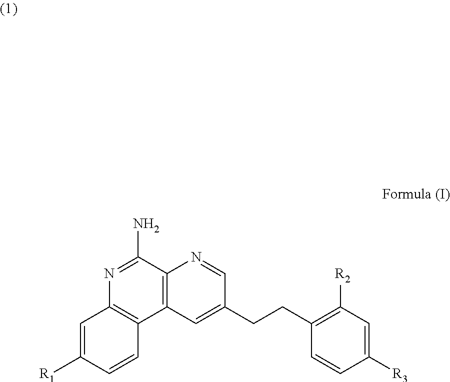

wherein:
   R¹ is H, C1-C6alkyl, —C(R⁵)₂OH, —L¹R⁵, —L¹R⁶, —L²R⁵, —L²R⁶, —OL R⁵, or —OL-R⁶;
   L¹ is —C(O)— or —O—;
   L² is C1-C6alkylene, C2-C6alkenylene, arylene, heteroarylene or —((CR⁴R⁴)ₚO)_{q}(CH₂)ₚ— wherein the C1-C6alkylene and C2-C6alkenylene of L² are optionally substituted with 1 to 4 fluoro groups;
   each L³ is independently selected from C1-C6alkylene and —((CR⁴R⁴)ₚO), (CH₂)ₚ— wherein the C1-C6alkylene of L³ is optionally substituted with 1 to 4 fluoro groups;
   L⁴ is arylene or heteroarylene;
   R² is H or C1-C6alkyl;
   R³ is selected from C1-C4alkyl, —L³R⁵, —L¹R⁵, —L³R⁷, —L³L⁴L³R⁷, —L³L⁴R⁵, —L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵, and —C(R⁵)₂OH;
   each R⁴ is independently selected from H and fluoro;
   R⁵ is —P(O)(OR⁹) 2,
   R⁶ is —CF₂P(O)(OR⁹)₂ or —C(O)OR¹⁰;
   R⁷ is —CF₂P(O)(OR⁹)₂ or —C(O)OR¹⁰;
   R⁸ is H or C1-C4alkyl;
   each R⁹ is independently selected from H and C1-C6alkyl;
   R¹⁰ is H or C1-C4alkyl;
   each p is independently selected from 1, 2, 3, 4, 5 and 6, and
   q is 1, 2, 3 or 4;
   or a pharmaceutically acceptable salt thereof.
11. The immunogenic composition or method of aspect 10, wherein the benzonapthyridine compound comprises or consists of the compound of formula (I), wherein:
   R¹ is —L²R⁶;
   R² is C1-C6alkyl;
   R³ is —OL³R⁵ or -OL³R⁷;
   R⁵ is —P(O)(OH)₂;
   R⁶ is —C(O)OH;
   R⁷ is —CF₂P(O)(OH)₂;
   L² is C1-C6alkylene;
   L³ is —((CR⁴R⁴)ₚO)_{q}(CH₂)ₚ—;
   R⁴ is H;
   q is 1 or 2; and
   p is 2;
or a pharmaceutically acceptable salt thereof.
12. The immunogenic composition or method of aspect 10 or 11, wherein the benzonapthyridine compound comprises or consists of the compound of formula (I), wherein:
   R¹ is —(CH₂)₂C(O) OH;
   R² is C1 alkyl; and
   R³ is —O(((CH₂)₂) O)₂ (CH₂)₂—P(O)(OH) 2;
or a pharmaceutically acceptable salt thereof.
13. The immunogenic composition or method of any one of aspects 10 to 12, wherein the benzonapthyridine compound is an arginine salt.
14. The immunogenic composition or method of any one of the preceding aspects, wherein the benzonapthyridine compound and/or the TLR7 agonist is LHD153R.
15. The immunogenic composition or method of any one of aspects 3 to 14, wherein the at least one antigen comprises at least one Group A Streptococcus antigen.
16. The immunogenic composition or method of any one of aspects 1, 2 or 7 to 15, wherein the at least one Group A Streptococcus antigen comprises at least one recombinant polypeptide.
17. The immunogenic composition or method of any one of aspects 1, 2 or 7 to 16, wherein the at least one Group A Streptococcus antigen comprises a polysaccharide-protein conjugate.
18. The immunogenic composition or method of aspect 17, wherein the ratio of the concentration (w/v) of the polysaccharide in the polysaccharide-protein conjugate to the concentration (w/v) of the TLR7 agonist or benzonapthyridine compound in the immunogenic composition is between 1:0.01 and 1:50, between 1:0.1 and 1:20, between 1:0.1 and 1:10, between 1:0.5 and 1:10, between 0.75:1 and 1:5, between 1:1 and 1:3, between 1:1.5 and 1:2.5, or around 1:2.
19. The immunogenic composition or method of aspect 18, wherein the ratio of the concentration (w/v) of the polysaccharide in the polysaccharide-protein conjugate to the concentration (w/v) of the TLR7 agonist or benzonapthyridine compound in the immunogenic composition is between 1:1.5 and 1:2.5.
20. The immunogenic composition or method of any one of aspects 17 to 19, wherein the ratio of the concentration (w/v) of the polysaccharide in the polysaccharide-protein conjugate to the concentration (w/v) of aluminium in the immunogenic composition is between 0.25:1 and 1:250, between 1:25 and 1:200, between 1:5 and 1:100, between 0.75:10 and 1:50, between 1:10 and 1:30, between 1:15 and 1:25, or around 1:21.
21. The immunogenic composition or method of any one of aspects 17 to 20, wherein the ratio of the concentration (w/v) of the polysaccharide in the polysaccharide-protein conjugate to the concentration (w/v) of aluminium in the immunogenic composition is between 1:15 and 1:25.
22. The immunogenic composition or method of any one of aspects 17 to 21, wherein the polysaccharide of the polysaccharide-protein conjugate is GAC.
23. The immunogenic composition or method of any one of aspects 17 to 22, wherein the protein of the polysaccharide-protein conjugate is CRM197, a SpyCEP polypeptide, a SLO polypeptide, or a SpyAD polypeptide.
24. The immunogenic composition or method of any one of aspects 17 to 23, wherein the protein of the polysaccharide-protein conjugate is CRM197.
25. The immunogenic composition or method of any one of aspects 1, 2, or 7 to 24, wherein the at least one Group A Streptococcus antigen comprises a SpyCEP polypeptide.
26. The immunogenic composition or method of aspect 25, wherein the ratio of the concentration (w/v) of the SpyCEP polypeptide to the concentration (w/v) of the TLR7 agonist or benzonapthyridine compound in the immunogenic composition is between 1:0.1 and 1:50, between 1:0.1 and 1:20, between 1:0.1 and 1:10, between 1:0.5 and 1:10, between 0.75:1 and 1:5, between 0.75:1 and 1:3, between 0.75.1 and 1:2.5, or around 1:1.
27. The immunogenic composition or method of aspect 26, wherein the ratio of the concentration (w/v) of the SpyCEP polypeptide to the concentration (w/v) of the TLR7 agonist or benzonapthyridine compound in the immunogenic composition is between 0.75:1 and 1:2.5.
28. The immunogenic composition or method of any one of aspects 25 to 27, wherein the ratio of the concentration (w/v) of the SpyCEP polypeptide to the concentration (w/v) of aluminium in the immunogenic composition is between 0.25:1 and 1:250, between 1:25 and 1:200, between 1:5 and 1:100, between 0.75:10 and 1:50, between 0.75:10 and 1:30, or between 1:5 and 1:15 or around 1:11.
29. The immunogenic composition or method of any one of aspects 25 to 28, wherein the ratio of the concentration (w/v) of the SpyCEP polypeptide to the concentration (w/v) of aluminium in the immunogenic composition is between 1:5 and 1:15.
30. The immunogenic composition or method any one of aspects 23 or 25 to 29, wherein the SpyCEP polypeptide does not comprise aspartate at a position corresponding to position 151 and/or serine at a position corresponding to position 617.
31. The immunogenic composition or method of any one of aspects 23 or 25 to 30, wherein the SpyCEP polypeptide comprises alanine at a position corresponding to position 151 and/or alanine at a position corresponding to position 617.
32. The immunogenic composition or method of any one of aspects 23 or 25 to 31, wherein the SpyCEP polypeptide comprises:
   (i) an amino acid sequence that is at least 90%, at least 95%, at least 98%, at least 99%, or 100% identical to a contiguous fragment of at least 1000, at least 1200, at least 1400, at least 1500, or at least 1550 amino acids of SEQ ID NO: 1 or 2; or
   (ii) an amino acid sequence that is at least 90%, at least 95%, at least 98%, at least 99%, or 100% identical to SEQ ID NO: 1 or 2; or
   (iii) an amino acid sequence of SEQ ID NO: 1 or 2.
33. The immunogenic composition or method of aspect 32, wherein the SpyCEP polypeptide comprises an amino acid sequence that is at least 98%, at least 99%, or 100% identical to a contiguous fragment of at least 1400, at least 1500, or at least 1550 amino acids of SEQ ID NO: 1 or 2.
34. The immunogenic composition or method of aspect 32, wherein the SpyCEP polypeptide comprises an amino acid sequence that is at least 98%, at least 99%, or 100% identical to SEQ ID NO: 1 or 2.
35. The immunogenic composition or method of aspect 32, wherein the SpyCEP polypeptide comprises an amino acid sequence of SEQ ID NO: 1 or 2.
36. The immunogenic composition or method of any one of aspects 1, 2, or 7 to 35, wherein the at least one Group A Streptococcus antigen comprises a SpyAD polypeptide.
37. The immunogenic composition or method of aspect 36, wherein the ratio of the concentration (w/v) of the SpyAD polypeptide to the concentration (w/v) of the TLR7 agonist or benzonapthyridine compound in the immunogenic composition is between 1:0.1 and 1:50, between 1:0.1 and 1:20, between 1:0.1 and 1:10, between 1:0.5 and 1:10, between 0.75:1 and 1:5, between 0.75:1 and 1:3, between 0.75.1 and 1:2.5, or around 1:1.
38. The immunogenic composition or method of aspect 37, wherein the ratio of the concentration (w/v) of the SpyAD polypeptide to the concentration (w/v) of the TLR7 agonist or benzonapthyridine compound in the immunogenic composition is between 0.75:1 and 1:2.5.
39. The immunogenic composition or method of any one of aspects 36 to 38, wherein the ratio of the concentration (w/v) of the SpyAD polypeptide to the concentration (w/v) of aluminium in the immunogenic composition is between 0.25:1 and 1:250, between 1:25 and 1:200, between 1:5 and 1:100, between 0.75:10 and 1:50, between 0.75:10 and 1:30, between 1:5 and 1:15 or around 1:11.
40. The immunogenic composition or method of any one of aspects 36 to 39, wherein the ratio of the concentration (w/v) of the SpyAD polypeptide to the concentration (w/v) of aluminium in the immunogenic composition is between 1:5 and 1:15.
41. The immunogenic composition or method of any one of aspects 23 or 36 to 40, wherein the SpyAD polypeptide comprises:
   (i) an amino acid sequence that is at least 90%, at least 95%, at least 98%, at least 99%, or 100% identical to a contiguous fragment of at least 600, at least 650, at least 700, at least 750, at least 770, or at least 800 amino acids of SEQ ID NO: 3 or 4; or
   (ii) an amino acid sequence that is at least 90%, at least 95%, at least 98%, at least 99%, or 100% identical to SEQ ID NO: 3 or 4; or
   (iii) an amino acid sequence of SEQ ID NO: 3 or 4.
42. The immunogenic composition or method of aspect 41, wherein the SpyAD polypeptide comprises an amino acid sequence that is at least 98%, at least 99%, or 100% identical to a contiguous fragment of at least 700, at least 750, or at least 800 amino acids of SEQ ID NO: 3 or 4.
43. The immunogenic composition or method of aspect 41, wherein the SpyAD polypeptide comprises an amino acid sequence that is at least 98%, at least 99%, or 100% identical to SEQ ID NO: 3 or 4.

44. The immunogenic composition or method of aspect 41, wherein the SpyAD polypeptide comprises an amino acid sequence of SEQ ID NO: 3 or 4.
45. The immunogenic composition or method of any one of aspects 1, 2, or 7 to 44, wherein the at least one Group A Streptococcus antigen comprises a SLO polypeptide.
46. The immunogenic composition or method of aspect 45, wherein the ratio of the concentration (w/v) of the SLO polypeptide to the concentration (w/v) of the TLR7 agonist or benzonapthyridine compound in the immunogenic composition is between 1:0.1 and 1:50, between 1:0.1 and 1:20, between 1:0.1 and 1:10, between 1:0.5 and 1:10, between 0.75:1 and 1:5, between 0.75:1 and 1:3, between 0.75.1 and 1:2.5, or around 1:1.
47. The immunogenic composition or method of aspect 46, wherein the ratio of the concentration (w/v) of the SLO polypeptide to the concentration (w/v) of the TLR7 agonist or benzonapthyridine compound in the immunogenic composition is between 0.75:1 and 1:2.5.
48. The immunogenic composition or method of any one of aspects 45 to 47, wherein the ratio of the concentration (w/v) of the SLO polypeptide to the concentration (w/v) of aluminium in the immunogenic composition is between 0.25:1 and 1:250, between 1:25 and 1:200, between 1:5 and 1:100, between 0.75:10 and 1:50, between 0.75:10 and 1:30, between 1:5 and 1:15, or around 1:11.
49. The immunogenic composition or method of any one of aspects 45 to 48, wherein the ratio of the concentration (w/v) of the SLO polypeptide to the concentration (w/v) of aluminium in the immunogenic composition is between 1:5 and 1:15.
50. The immunogenic composition or method of any one of aspects 23 or 45 to 49, wherein the SLO polypeptide does not comprise proline at a position corresponding to position 427 and/or tryptophan at a position corresponding to position 535.
51. The immunogenic composition or method of any one of aspects 23 or 45 to 50, wherein the SLO polypeptide comprises leucine at a position corresponding to position 427 and/or phenylalanine at a position corresponding to position 535.
52. The immunogenic composition or method of any one of aspects 23 or 45 to 51, wherein the SLO polypeptide comprises:
   (i) an amino acid sequence that is at least 90%, at least 95%, at least 98%, at least 99%, or 100% identical to a contiguous fragment of at least 350, at least 400, at least 450, at least 500, or at least 530 amino acids of SEQ ID NO: 5 or 6; or
   (ii) an amino acid sequence that is at least 90%, at least 95%, at least 98%, at least 99%, or 100% identical to SEQ ID NO: 5 or 6; or
   (iii) an amino acid sequence of SEQ ID NO: 5 or 6.
53. The immunogenic composition or method of aspect 52, wherein the SLO polypeptide comprises an amino acid sequence that is at least 98%, at least 99%, or 100% identical to a contiguous fragment of at least 450, at least 500, or at least 530 amino acids of SEQ ID NO: 5 or 6.
54. The immunogenic composition or method of aspect 52, wherein the SLO polypeptide comprises an amino acid sequence that is at least 98%, at least 99%, or 100% identical to SEQ ID NO: 5 or 6.
55. The immunogenic composition or method of aspect 52, wherein the SLO polypeptide comprises an amino acid sequence of SEQ ID NO: 5 or 6.
56. The immunogenic composition or method of any one of the preceding aspects, wherein at least 50%, at least 75%, or at least 80% of the at least one antigen or the at least one Group A Streptococcus antigen in the immunogenic composition is adsorbed to the aluminium salt.
57. The immunogenic composition or method of any one of the preceding aspects, wherein at least 50%, at least 75%, or at least 80% of the TLR7 agonist and/or benzonapthyridine compound is adsorbed to the aluminium salt.
58. The immunogenic composition or method of any one of the preceding aspects, wherein the immunogenic composition is more immunogenic compared to an equivalent immunogenic composition that does not comprise the TLR7 agonist and/or the benzonapthyridine compound.
59. The immunogenic composition or method of any one of the preceding aspects, wherein the immunogenic composition raises a greater number of antibodies when administered to mice compared to an equivalent immunogenic composition that does not comprise the TLR7 agonist and/or the benzonapthyridine compound.
60. The immunogenic composition or method of aspect 59, wherein the immunogenic composition raises a greater number of antibodies against at least one of the following antigens:
   (i) SpyCEP;
   (ii) SpyAD;
   (iii) SLO; and/or
   (iv) GAC,
      when administered to mice compared to an equivalent immunogenic composition that does not comprise the TLR7 agonist and/or the benzonapthyridine compound.
61. The immunogenic composition or method of aspect 60, wherein the immunogenic composition raises a greater number of antibodies against all of the following antigens SpyCEP, SpyAD, SLO and GAC when administered to mice compared to an equivalent immunogenic composition that does not comprise the TLR7 agonist and/or the benzonapthyridine compound.
62. The immunogenic composition or method of any one of aspects 59 to 61, wherein the number of antibodies is measured using a fluorescence immunoassay in which the antigens are conjugated to beads and beads comprising complexes of the antigens and antibodies are detected by measuring fluorescence.
63. The immunogenic composition or method of any one of the preceding aspects, wherein the immunogenic composition raises a greater number of functional antibodies when administered to mice compared to an equivalent immunogenic composition that does not comprise the TLR7 agonist and/or the benzonapthyridine compound.
64. The immunogenic composition or method of aspect 63, wherein the immunogenic composition raises a greater number of functional antibodies against:
   (i) SpyCEP; and/or
   (ii) SLO,
      when administered to mice compared to an equivalent immunogenic composition that does not comprise the TLR7 agonist and/or the benzonapthyridine compound.
65. The immunogenic composition or method of aspect 64, wherein the immunogenic composition raises a greater number of functional antibodies against SpyCEP and SLO when administered to mice compared to an equivalent immunogenic composition that does not comprise the TLR7 agonist and/or the benzonapthyridine compound.
66. The immunogenic composition or method of any one of aspects 1 to 65, wherein the immunogenic composition further comprises a pharmaceutically acceptable excipient.
67. A vaccine comprising the immunogenic composition of any one of aspects 1, 2, or 4 to 66.
68. The immunogenic composition of any one of aspects 1, 2 or 4 to 66 or the vaccine of aspect 67 for use in a method of preventing infection by and/or preventing autoimmune disease following infection by Group A Streptococcus.
69. A method of preventing infection by and/or preventing autoimmune disease following infection by Group A Streptococcus comprising administering an effective amount of the immunogenic composition of aspects 1, 2, or 4 to 66 or the vaccine of aspect 67 to a subject.
70. Use of the immunogenic composition of any one of aspects 1, 2 or 4 to 66 or the vaccine of aspect 67 for preparing a medicament for use in a method of preventing infection by and/or preventing autoimmune disease following infection by Group A Streptococcus.

1. (canceled)

2. An immunogenic composition comprising:
(a) Group A Streptococcus SLO, SpyAD, and SpyCEP antigens;
(b) an aluminium salt;
(c) a polysaccharide-protein conjugate, wherein the polysaccharide of the polysaccharide protein conjugate is GAC and the protein of the polysaccharide-protein conjugate is CRM197; and
(d) around 0.1 mg/ml of a benzonapthyridine compound of Formula (I) 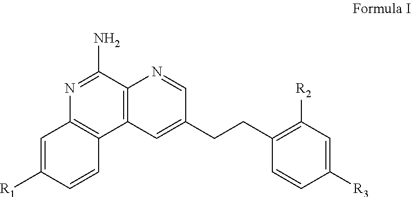
wherein:
R¹ is H, C1-C6alkyl, —C(R⁵)₂OH, —L¹R⁵-L¹R⁶-L²R⁵, —L²R⁶, —OL²R⁵ or -OL²R⁶; L¹ is —C(O)— or —O—;
L² is C1-C6alkylene, C2-C6alkenylene, arylene, heteroarylene or —((CR⁴R⁴)ₚO)_{q}(CH₂)ₚ— wherein the C1-C6alkylene and C2-C6alkenylene of L² are optionally substituted with 1 to 4 fluoro groups;
each L³ is independently selected from C1-C6alkylene and —((CR⁴R⁴)ₚO)_{q}(CH₂)ₚ— wherein the C1-C6alkylene of L³ is optionally substituted with 1 to 4 fluoro groups;
L⁴ is arylene or heteroarylene;
R² is H or C1-C6alkyl;
R³ is selected from C1-C4alkyl, —L³R⁵, —L¹R⁵, —L³R⁷, —L³L⁴L³R⁷, —L³L⁴R⁵, —L³L⁴L³R⁵-OL³R⁵, —OL³R⁷, —OL³L⁴R⁷, —OL³L⁴L³R⁷, —OR⁸, —OL³L⁴R⁵, —OL³L⁴L³R⁵, and —C(R⁵)₂OH;
each R⁴ is independently selected from H and fluoro;
R⁵ is —P(O)(OR⁹)₂,
R⁶ is —CF₂P(O)(OR)₂ or —C(O)OR¹⁰;
R⁷ is —CF₂P(O)(OR⁹)₂ or —C(O)OR¹⁰;
R⁸ is H or C1-C4alkyl;
each R^{o} is independently selected from H and C1-C6alkyl;
R¹⁰ is H or C1-C4alkyl;
each p is independently selected from 1, 2, 3, 4, 5 and 6, and
q is 1, 2, 3 or 4;
or a pharmaceutically acceptable salt thereof.

3. A method of making the immunogenic composition of claim 2 comprising preparing a mixture of:
iii) the at least one Group A Streptococcus antigen pre-absorbed on the aluminium salt and the benzonapthyridine compound of Formula (I) pre-absorbed on the aluminium salt.

4. (canceled)

5. The immunogenic composition of claim 2, wherein the aluminium salt comprises or consists of aluminium phosphate or aluminium hydroxide.

6. The immunogenic composition of claim 2, wherein the aluminium salt comprises or consists of aluminium hydroxide.

7. The immunogenic composition of claim 2, wherein R¹ is —(CH₂)₂C(O)OH;
R² is C1 alkyl; and
R³ is —O(((CH₂)₂)O)₂ (CH-₂)₂—P(O)(OH)₂.

8.-9. (canceled)

10. The immunogenic composition of claim 2, wherein the SpyCEP antigen is a SpyCEP polypeptide comprising alanine at a position corresponding to position 151 and/or alanine at a position corresponding to position 617.

11. The immunogenic composition of claim 10, wherein the SpyCEP polypeptide comprises an amino acid sequence that is at least 98%, at least 99%, or 100% identical to SEQ ID NO: 1 or 2.

12. (canceled)

13. The immunogenic composition- or method of claim 12 of claim 2, wherein the Spy AD antigen is a SpyAD polypeptide comprising an amino acid sequence that is at least 98%, at least 99%, or 100% identical to SEQ ID NO: 3 or 4.

14. (canceled)

15. The immunogenic composition 14 of claim 2, wherein the SLO antigen is a SLO polypeptide comprising leucine at a position corresponding to position 427 and/or phenylalanine at a position corresponding to position 535.

16. The immunogenic composition of claim 15, wherein the SLO polypeptide comprises an amino acid sequence that is at least 98%, at least 99%, or 100% identical to SEQ ID NO: 5 or 6.

17. The immunogenic composition of claim 2, wherein the immunogenic composition is more immunogenic compared to an equivalent immunogenic composition that does not comprise the benzonapthyridine compound.

18. The immunogenic composition of claim 2, wherein the immunogenic composition raises a greater number of antibodies when administered to mice compared to an equivalent immunogenic composition that does not comprise the benzonapthyridine compound.

19. The immunogenic composition of claim 18, wherein the immunogenic composition raises a greater number of antibodies against SpyCEP, SpyAD, SLO and GAC when administered to mice compared to an equivalent immunogenic composition that does not comprise the benzonapthyridine compound.

20. The immunogenic composition of claim 2, wherein the immunogenic composition raises a greater number of functional antibodies against:
(i) SpyCEP; and/or
(ii) SLO,
when administered to mice compared to an equivalent immunogenic composition that does not comprise the benzonapthyridine compound.

21. The immunogenic composition of claim 2, wherein the immunogenic composition further comprises a pharmaceutically acceptable excipient.

22.-24. (canceled)

25. A method of raising antibodies to Group A Streptococcus in a human comprising administering an immunologically effective amount of the immunogenic composition of claim 2 to a human.

26. The immunogenic composition of claim 2 wherein the benzonapthyridine compound of Formula (1) is selected from the group consisting of 4-(4-(2-(5-amino-8-methylbenzo[f][1,7]naphthyridin-2-yl)ethyl)-3-methylphenoxy)-1,1-difluorobutylphosphonic acid;
3-(5-amino-2-(4-(4,4-difluoro-4-phosphonobutoxy)-2-methylphenethyl)benzo[f][1,7]naphthyridin-8-yl)propanoic acid;
3-(5-amino-2-(4-(2-(3, 3-difluoro-3-phosphonopropoxy)ethoxy)-2-methylphenethyl)benzo[f][1,7]naphthyridin-8-yl)propanoic acid;
3-(5-amino-2-(2-methyl-4-(2-(2-(2-phosphonoethoxy)ethoxy)ethoxy)phenethyl)benzo[f][1,7]naphthyridin-8-yl)propanoic acid;
4-(2-(5-amino-8-methylbenzo[f][1,7]naphthyridin-2-yl)ethyl)-3-methylphenyl dihydrogen phosphate;
(4-(2-(5-amino-8-methylbenzo[f][1,7]naphthyridin-2-yl)ethyl)-3-methylphenoxy)methylphosphonic acid;
5-(4-(2-(5-amino-8-methylbenzo[f][1,7]naphthyridin-2-yl)ethyl)-3-methylphenoxy)-1,1-difluoropentylphosphonic acid;
4-(4-(2-(5-amino-8-methylbenzo[f][1,7]naphthyridin-2-yl)ethyl)-3-methylphenoxy)-1,1-difluorobutylphosphonic acid;
3-(2-(4-(2-(5-amino-8-methylbenzo[f][1,7]naphthyridin-2-yl)ethyl)-3-methylphenoxy) ethoxy)-1,1-difluoropropylphosphonic acid;
2-(4-((4-(2-(5-amino-8-methylbenzo[f][1,7]naphthyridin-2-yl)ethyl)-3-methylphenoxy)methyl)phenyl)-1,1-difluoroethylphosphonic acid;
2-(5-amino-2-(4-methoxy-2-methylphenethyl)benzo[f][1,7]naphthyridin-8-yl)-1,1-difluoro-2-oxoethylphosphonic acid;
(E)-2-(5-amino-2-(4-methoxy-2-methylphenethyl)benzo[f][1,7]naphthyridin-8-yl)vinylphosphonic acid;
2-(5-amino-2-(4-methoxy-2-methylphenethyl)benzo[f][1,7]naphthyridin-8-yl)ethylphosphonic acid;
(E)-2-(5-amino-2-(4-methoxy-2-methylphenethyl)benzo[f][1,7]naphthyridin-8-yl)-1-fluorovinylphosphonic acid;
3-((4-(2-(5-amino-8-methylbenzo[f][1,7]naphthyridin-2-yl)ethyl)-3-methylphenoxy)methyl)phenylphosphonic acid;
5-amino-2-(4-methoxy-2-methylphenethyl)benzo[f][1,7]naphthyridine-8-carbonylphosphonic acid;
3-(5-amino-2-(2-methyl-4-(3-phosphonopropoxy)phenethyl)benzo[f][1,7]naphthyridin-8-yl)propanoic acid;
(4-(2-(5-amino-8-methylbenzo[f][1,7]naphthyridin-2-yl)ethyl)-3-methylphenyl)(hydroxy)methylenediphosphonic acid;
3-(5-amino-2-(4-(2-(2-(3,3-difluoro-3-phosphonopropoxy)ethoxy)ethoxy)-2-methylphenethyl)benzo[f][1,7]naphthyridin-8-yl)propanoic acid;
(5-amino-2-(4-methoxy-2-methylphenethyl)benzo[f][1,7]naphthyridin-8-yl)(hydroxy)methylenediphosphonic acid;
3-(5-amino-2-(2-methyl-4-(2-(2-phosphonoethoxy)ethoxy)phenethyl)benzo[f][1,7] naphthyridin-8-yl)propanoic acid;
2-(4-(2-(5-amino-8-methylbenzo[f][1,7]naphthyridin-2-yl)ethyl)-3-methylphenoxy)ethylphosphonic acid;
6-(4-(2-(5-amino-8-methylbenzo[f][1,7]naphthyridin-2-yl)ethyl)-3-methylphenoxy) hexylphosphonic acid;
6-(4-(2-(5-amino-8-methylbenzo[f][1,7]naphthyridin-2-yl)ethyl)-3-methylphenoxy)-1,1-difluorohexylphosphonic acid;
4-((4-(2-(5-amino-8-methylbenzo[f][1,7]naphthyridin-2-yl)ethyl)-3-methylphenoxy)methyl)benzylphosphonic acid;
2-(2-(2-(4-(2-(5-amino-8-methylbenzo[f][1,7]naphthyridin-2-yl)ethyl)-3-methylphenoxy) ethoxy)ethoxy)ethylphosphonic acid;
3-[5-amino-2-(2-[4-[2-(3,3-difluoro-3-phosphonopropoxy) ethoxy]-2-methylphenyl]ethyl)benzo[f]1,7-naphthyridin-8-yl]propanoic acid;
[5-[4-(2-{5-amino-8-methylbenzo[f]1,7-naphthyridin-2-yl)ethyl)-3-methylphenoxy]pentyl)phosphonic acid;
(4-[4-(2-(5-amino-8-methylbenzo[f]1,7-naphthyridin-2-yl}ethyl)-3-methylphenoxy]butyl}phosphonic acid;
and pharmaceutically acceptable salts thereof.

27. The immunogenic composition of claim 2 wherein the benzonapthyridine compound of Formula (I) is an arginine salt of 3-(5-amino-2-(2-methyl-4-(2-(2-(2-phosphonoethoxy)ethoxy)ethoxy)phenethyl)benzo[f][1,7]naphthyridin-8-yl)propanoic acid.

28. The immunogenic composition of claim 2 wherein the benzonapthyridine compound of Formula (I) is an L-arginine salt of 3-(5-amino-2-(2-methyl-4-(2-(2-(2-phosphonoethoxy)ethoxy)ethoxy)phenethyl)benzo[f][1,7]naphthyridin-8-yl)propanoic acid.


SEQUENCE LISTING (7 sequences)

SEQ ID NO: 1 - protein, 1581 aa, synthetic construct
MADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIATGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTAMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKNAQGNPVLAISPNGDNNQDFAAFKGVFLRKYQGLKASVYHASDKEHKNPLWVSPESFKGDKNFNSDIRFAKSTTLLGTAFSGKSLTGAELPDGHYHYVVSYYPDVVGAKRQEMTFDMILDRQKPVLSQATFDPETNRFKPEPLKDRGLAGVRKDSVFYLERKDNKPYTVTINDSYKYVSVEDNKTFVERQADGSFILPLDKAKLGDFYYMVEDFAGNVAIAKLGDHLPQTLGKTPIKLKLTDGNYQTKETLKDNLEMTQSDTGLVTNQAQLAVVHRNQPQSQLTKMNQDFFISPNEDGNKDFVAFKGLKNNVYNDLTVNVYAKDDHQKQTPIWSSQAGASVSAIESTAWYGITARGSKVMPGDYQYVVTYRDEHGKEHQKQYTISVNDKKPMITQGRFDTINGVDHFTPDKTKALDSSGIVREEVFYLAKKNGRKFDVTEGKDGITVSDNKVYIPKNPDGSYTISKRDGVTLSDYYYLVEDRAGNVSFATLRDLKAVGKDKAVVNFGLDLPVPEDKQIVNFTYLVRDADGKPIENLEYYNNSGNSLILPYGKYTVELLTYDTNAAKLESDKIVSFTLSADNNFQQVTFKITMLATSQITAHFDHLLPEGSRVSLKTAQDQLIPLEQSLYVPKAYGKTVQEGTYEVVVSLPKGYRIEGNTKVNTLPNEVHELSLRLVKVGDASDSTGDHKVMSKNNSQALTASATPTKSTTSATAKA

SEQ ID NO: 2 - protein, 1647 aa, Streptococcus pyogenes
MEKKQRFSLRKYKSGTFSVLIGSVFLVMTTTVAADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIDTGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTSMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKNAQGNPVLAISPNGDNNQDFAAFKGVFLRKYQGLKASVYHASDKEHKNPLWVSPESFKGDKNFNSDIRFAKSTTLLGTAFSGKSLTGAELPDGHYHYVVSYYPDVVGAKRQEMTFDMILDRQKPVLSQATFDPETNRFKPEPLKDRGLAGVRKDSVFYLERKDNKPYTVTINDSYKYVSVEDNKTFVERQADGSFILPLDKAKLGDFYYMVEDFAGNVAIAKLGDHLPQTLGKTPIKLKLTDGNYQTKETLKDNLEMTQSDTGLVTNQAQLAVVHRNQPQSQLTKMNQDFFISPNEDGNKDFVAFKGLKNNVYNDLTVNVYAKDDHQKQTPIWSSQAGASVSAIESTAWYGITARGSKVMPGDYQYVVTYRDEHGKEHQKQYTISVNDKKPMITQGRFDTINGVDHFTPDKTKALDSSGIVREEVFYLAKKNGRKFDVTEGKDGITVSDNKVYIPKNPDGSYTISKRDGVTLSDYYYLVEDRAGNVSFATLRDLKAVGKDKAVVNFGLDLPVPEDKQIVNFTYLVRDADGKPIENLEYYNNSGNSLILPYGKYTVELLTYDTNAAKLESDKIVSFTLSADNNFQQVTFKITMLATSQITAHFDHLLPEGSRVSLKTAQDQLIPLEQSLYVPKAYGKTVQEGTYEVVVSLPKGYRIEGNTKVNTLPNEVHELSLRLVKVGDASDSTGDHKVMSKNNSQALTASATPTKSTTSATAKALPSTGEKMGLKLRIVGLVLLGLTCVFSRKKSTKD

SEQ ID NO: 3 - protein, 824 aa, Streptococcus pyogenes
MSVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 4 - protein, 873 aa, synthetic construct
MDLEQTKPNQVKQKIALTSTIALLSASVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQRALKAGVVMLAAVGLTGFRFRKESK

SEQ ID NO: 5 - protein, 543 aa, synthetic construct
MASESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNLAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAFEWWRKVIDERDVKLSKEINVNISGSTLSPYGSITYK

SEQ ID NO: 6 - protein, 571 aa, Streptococcus pyogenes
MSNKKTFKKYSRVAGLLTAALIIGNLVTANAESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 7 - protein, 535 aa, synthetic construct
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS